(19) United States
(12) Patent Application Publication
(10) Pub. No.: US 2011/0114185 A1
(43) Pub. Date: May 19, 2011

(54) PHOTOELECTRIC CONVERSION ELEMENT

(75) Inventor: Takehito Kato, Oyama-shi (JP)

(73) Assignee: SUMITOMO CHEMICAL COMPANY, LIMITED, Chuo-ku, Tokyo (JP)

(21) Appl. No.: 13/003,741

(22) PCT Filed: Jul. 30, 2009
(86) PCT No.: PCT/JP2009/063904

§ 371 Date: Jan. 11, 2011

(30) Foreign Application Priority Data
Jul. 31, 2008 (JP) 2008-197559

(51) Int. Cl. H01L 51/46

(52) U.S. Cl. 136/263

(57) ABSTRACT

A photoelectric conversion element comprising a first electrode, a second electrode, a functional layer arranged between the first electrode and the second electrode, and a porous semiconductor material arranged between the first electrode and the functional layer. The functional layer contains a polymer compound having an aromatic amine residue, and a dye is adsorbed on the porous semiconductor material. The photoelectric conversion element may additionally have a dense layer between the first electrode and the functional layer, and a dye may be absorbed on a part of the functional-layer-side surface of the dense layer. The photoelectric conversion element may additionally have an organic layer between the functional layer and the second electrode.

### TECHNICAL FIELD

The present invention relates to a photoelectric conversion element.

### BACKGROUND ART

A dye-sensitized solar battery has been developed as one of photoelectric conversion elements. The dye-sensitized solar battery comprises first and second electrodes for taking out an electric power, a semiconductor material containing a dye adsorbed thereon, and a charge transport layer provided to cover the semiconductor material. In a dye-sensitized solar battery in which an iodine solution is used in a charge transport layer, one with a photoelectric conversion efficiency of more than 10% has been reported.

When an iodine solution is used in a charge transport layer, the electrode or the like may be corroded by iodine contained in the charge transport layer and deterioration of photoelectric conversion characteristics may be caused by volatilization of the iodine solution. Furthermore, since the charge transport layer is liquid, damage may cause leakage of a liquid contained in the charge transport layer, and thus photoelectric conversion efficiency may decrease.

Therefore, there has been proposed a dye-sensitized solar battery in which an electrically conductive polymer is used in the charge transport layer in place of the iodine solution. Use of poly(3-hexylthiophene) (P3HT), which is an electrically conductive polymer, in place of the iodine solution enables suppression of corrosion of the electrode or the like, and also enables suppression of deterioration of photoelectric conversion characteristics caused by volatilization. Further, since a solid charge transport layer is formed, leakage of a material contained in the charge transport layer can be suppressed, and thus a decrease in photoelectric conversion efficiency can be prevented. In such a manner, a dye-sensitized solar battery with high reliability has been realized by using the electrically conductive polymer in the charge transport layer (for example, ECS Transactions, 2006, Vol. 2, Issu. 12, pp. 145-154).

### DISCLOSURE OF THE INVENTION

However, in a dye-sensitized solar battery using a charge transport layer containing poly(3-hexylthiophene), sufficient photoelectric conversion efficiency is not necessarily achieved and it is desired to further improve the photoelectric conversion efficiency.

It is an object of the present invention to provide a photoelectric conversion element which is free from leakage of a liquid material and also has high photoelectric conversion efficiency.

First, the present invention provides a photoelectric conversion element comprising a first electrode; a second electrode; a functional layer between the first electrode and the second electrode, the functional layer containing a polymer compound having an aromatic amine residue; and a porous semiconductor material containing a dye adsorbed thereon between the first electrode and the functional layer.

Second, the present invention provides the photoelectric conversion element, which comprises a dense layer between the first electrode layer and the functional layer, wherein the porous semiconductor material containing a dye adsorbed thereon adheres onto a surface of the functional layer side of the dense layer.

Third, the present invention provides the photoelectric conversion element, which comprises an organic layer between the functional layer and the second electrode.

Fourth, the present invention provides the photoelectric conversion element, wherein the aromatic amine residue is one or more kinds of groups selected from the group consisting of a group in which at least one hydrogen atom has been removed from a structure represented by the formula (1), a group represented by the formula (5-1) and a group represented by the formula (5-2):

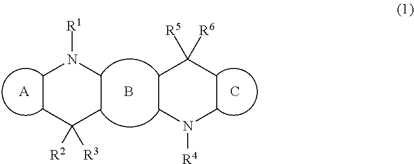

wherein ring A, ring B and ring C are the same or different and each represent an aromatic ring, R¹ and R⁴ are the same or different and each represent a monovalent group, and R², R³, R⁵ and R⁶ are the same or different and each represent a hydrogen atom or a monovalent group;

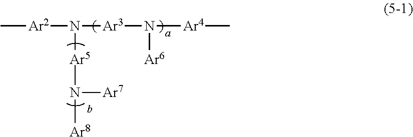

wherein Ar², Ar³, Ar⁴ and Ar⁵ are the same or different and each represent an arylene group or a divalent heterocyclic group, Ar⁶, Ar⁷ and Ar⁸ are the same or different and each represent an aryl group or a monovalent heterocyclic group, a and b are the same or different and each represent 0 or a positive integer, when plural Ar³(s) are present, they may be respectively the same or different, when plural Ar⁵(s) are present, they may be respectively the same or different, when plural Ar⁶(s) are present, they may be respectively the same or different, and when plural Ar⁷(s) are present, they may be respectively the same or different; and

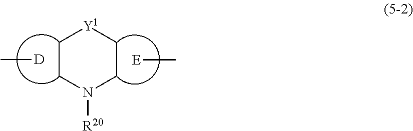

wherein ring D and ring E are the same or different and each represent an aromatic ring having a bond, Y¹ represents —O—, —S— or —C(═O)—, and R²⁰ represents a monovalent group.

Fifth, the present invention provides the photoelectric conversion element, wherein R², R³, R⁵ and R⁶ are hydrogen atoms, alkyl groups, aryl groups, arylalkyl groups, alkenyl groups or alkynyl groups.

Sixth, the present invention provides the photoelectric conversion element, wherein R², R³, R⁵ and R⁶ are groups represented by the formula (2):

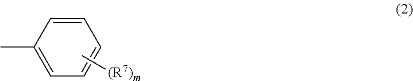

wherein R⁷ represents a halogen atom, an alkyl group, an alkoxy group, an alkylthio group, an aryl group, an aryloxy group, an arylthio group, an arylalkyl group, an arylalkyloxy group, an arylalkylthio group, an alkenyl group, an alkynyl group, a substituted amino group or a substituted silyl group, m represents ah integer of from 0 to 5 and, when m is 2 or more, plural R⁷(s) may be the same or different from each other.

Seventh, the present invention provides the photoelectric conversion element, wherein the group in which at least one hydrogen atom has been removed from a structure represented by the formula (1) is a group represented by the formula (3):

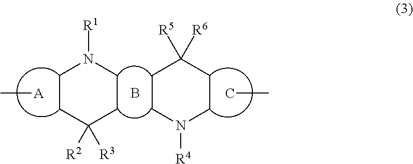

wherein ring A, ring B, ring C, R¹, R², R³, R⁴, R⁵ and R⁶ have the same meanings as defined above.

Eighth, the present invention provides the photoelectric conversion element, wherein the polymer compound contains a repeating unit represented by the formula (4):

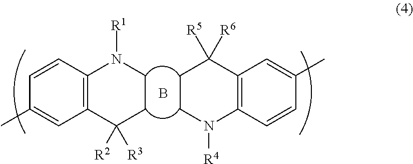

wherein ring B, R¹, R², R³, R⁴, R⁵ and R⁶ have the same meanings as defined above.

Ninth, the present invention provides the photoelectric conversion element, wherein the polymer compound contains the repeating unit represented by the formula (4) and the group represented by the formula (5-1) or the group represented by the formula (5-2).

Tenth, the present invention provides the photoelectric conversion element, wherein the polymer compound further contains a repeating unit represented by the formula (6):

—Ar¹—(CR⁸═CR⁹)—ₙ (6)

wherein Ar¹ represents an arylene group or a divalent heterocyclic group, R⁸ and R⁹ are the same or different and each represent a hydrogen atom, an alkyl group, an aryl group, a monovalent heterocyclic group or a cyano group, and n represents 0 or 1.

Eleventh, the present invention provides the photoelectric conversion element, wherein the polymer compound has a polystyrene-equivalent number average molecular weight of from 2×10³ to 1×10⁸.

Twelfth, the present invention provides the photoelectric conversion element, wherein the functional layer is in contact with the porous semiconductor material containing a dye adsorbed thereon.

Thirteenth, the present invention provides the photoelectric conversion element, wherein the highest occupied molecular orbital level of the polymer compound is higher than the highest occupied molecular orbital level of the dye.

Fourteenth, the present invention provides the photoelectric conversion element, wherein the lowest unoccupied molecular orbital level of the polymer compound is higher than the lowest unoccupied molecular orbital level of the dye.

Fifteenth, the present invention provides the photoelectric conversion element, wherein the polymer compound has a positive hole mobility of 1×10⁻⁴ cm²/Vsec or more.

Sixteenth, the present invention provides the photoelectric conversion element, wherein the first and/or second electrodes are made of at least one kind selected from the group consisting of an electrically conductive polymer, an oxide semiconductor material and a metal.

Seventeenth, the present invention provides the photoelectric conversion element, wherein at least a portion of the second electrode is in contact with the functional layer.

### BRIEF DESCRIPTION OF THE DRAWINGS

FIG. 1 is a diagram schematically showing a photoelectric conversion element 1 according to an embodiment of the present invention.

### DESCRIPTION OF REFERENCE NUMERALS

1: Photoelectric conversion element
2: Photoelectrode
3: Functional layer
4: Dense layer
5: First electrode
6: Substrate
7: Organic layer
8: Second electrode

### MODE FOR CARRYING OUT THE INVENTION

The present invention will be described in detail below.

A polymer compound contained in a functional layer of the photoelectric conversion-element of the present invention has an aromatic amine residue.

Examples of the functional layer include a layer having a charge transport function. The charge transport function includes electron transport properties, hole transporting properties, ion conductivity and the like.

The aromatic amine residue is a group in which at least one hydrogen atom has been removed from an aromatic amino compound. The aromatic amine residue is preferably one or more kinds of groups selected from the group consisting of a group in which at least one hydrogen atom has been removed from a structure (compound) represented by the formula (1), a group represented by the formula (5-1) and a group represented by the formula (5-2), and more preferably a group in which at least one hydrogen atom has been removed from a structure represented by the formula (1):

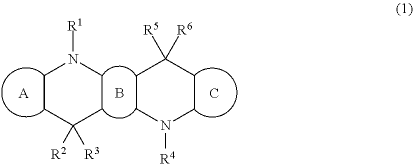

wherein ring A, ring B and ring C are the same or different and each represent an aromatic ring, R¹ and R⁴ are the same or different and each represent a monovalent group, and R², R³, R⁵ and R⁶ are the same or different and each represent a hydrogen atom or a monovalent group.

In the formula (1), ring A, ring B and ring C may have a substituent.

Examples of the aromatic ring represented by ring A, ring B and ring C include aromatic hydrocarbon rings such as a benzene ring, a naphthalene ring, an anthracene ring, a phenanthrene ring, a pyrene ring, a perylene ring, a tetracene ring, a pentacene ring, and a fluorene ring; and heteroaromatic rings such as a pyridine ring, a pyrimidine ring, a pyridazine ring, a pyrazine ring, a quinoline ring, an isoquinoline ring, a quinoxaline ring, a quinazoline ring, an acridine ring, a phenanthroline ring, a thiophene ring, a benzothiophene ring, a dibenzothiophene ring, a thiophene oxide ring, a benzothiophene oxide ring, a dibenzothiophene oxide ring, a furan ring, a benzofuran ring, a pyrrole ring, an indole ring, a dibenzopyrrole ring, a silole ring, a benzosilole ring, a dibenzosilole ring, a borole ring, a benzoborole ring, and a dibenzoborole ring. Among these rings, an aromatic hydrocarbon ring is preferred, a benzene ring, a naphthalene ring, an anthracene ring and a phenanthrene ring are more preferred, and a benzene ring is still more preferred, from the viewpoint of heat resistance of a polymer compound, characteristics of a photoelectric conversion element and the like.

Examples of the substituent, which may be included in ring A, ring B and ring C, include a halogen atom, an alkyl group, an alkoxy group, an alkylthio group, an aryl group, an aryloxy group, an arylthio group, an arylalkyl group, an arylalkoxy group, an arylalkylthio group, an alkenyl group, an alkynyl group, a substituted amino group, a substituted silyl group, an acyl group, an acyloxy group, an imine residue, an amide group, an acid imide group, a monovalent heterocyclic group, a substituted carboxyl group, a heteroaryloxy group, and a heteroarylthio group.

Examples of the halogen atom include a fluorine atom, a chlorine atom, a bromine atom, and an iodine atom.

The alkyl group may be linear or branched, or may also be a cycloalkyl group. Further, a hydrogen atom in the alkyl group may be substituted with a fluorine atom. The number of carbon atoms of the alkyl group is usually from about 1 to 30, and preferably from about 3 to 15 from the viewpoint of solubility of a polymer compound in a solvent. Examples of the alkyl group include a methyl group, an ethyl group, a propyl group, an i-propyl group, a butyl group, an i-butyl group, a s-butyl group, a t-butyl group, a pentyl group, an isoamyl group, a hexyl group, a cyclohexyl group, a heptyl group, an octyl group, a 2-ethylhexyl group, a nonyl group, a decyl group, a 3,7-dimethyloctyl group, a lauryl group, a trifluoromethyl group, a pentafluoroethyl group, a perfluorobutyl group, a perfluorohexyl group and a perfluorooctyl group. Among these groups, a pentyl group, an isoamyl group, a hexyl group, an octyl group, a 2-ethylhexyl group, a decyl group, and a 3,7-dimethyloctyl group are preferred from the viewpoint of a balance between solubility of a polymer compound in an organic solvent, photoelectric conversion element characteristics, ease of synthesis of a monomer and the like and heat resistance of a polymer compound.

The alkoxy group may be linear or branched, or may also be a cycloalkyloxy group. Further, a hydrogen atom in the alkoxy group may be substituted with a fluorine atom. The number of carbon atoms of the alkoxy group is usually from about 1 to 30, and preferably from about 3 to 15, from the viewpoint of solubility of a polymer compound in a solvent. Examples of the alkoxy group include a methoxy group, an ethoxy group, a propyloxy group, an i-propyloxy group, a butoxy group, an i-butoxy group, a s-butoxy group, a t-butoxy group, a pentyloxy group, a hexyloxy group, a cyclohexyloxy group, a heptyloxy group, an octyloxy group, a 2-ethylhexyloxy group, a nonyloxy group, a decyloxy group, a 3,7-dimethyloctyloxy group, a lauryloxy group, a trifluoromethoxy group, a pentafluoroethoxy group, a perfluorobutoxy group, a perfluorohexyl group, a perfluorooctyl group, a methoxymethyloxy group and a 2-methoxyethyloxy group. Among these groups, a pentyloxy group, a hexyloxy group, an octyloxy group, a 2-ethylhexyloxy group, a decyloxy group, and a 3,7-dimethyloctyloxy group are preferred from the viewpoint of a balance between solubility of a polymer compound in an organic solvent, photoelectric conversion element characteristics, ease of synthesis of a monomer and the like and heat resistance of a polymer compound.

The alkylthio group may be linear or branched, and also may be a cycloalkylthio group. Further, a hydrogen atom in the alkylthio group may be substituted with a fluorine atom. The number of carbon atoms of the alkylthio group is usually from about 1 to 30, and preferably from about 3 to 15, from the viewpoint of solubility of a polymer compound in a solvent. Examples of the alkylthio group include a methylthio group, an ethylthio group, a propylthio group, an i-propylthio group, a butylthio group, an i-butylthio group, a s-butylthio group, a t-butylthio group, a pentylthio group, a hexylthio group, a cyclohexylthio group, a heptylthio group, an octylthio group, a 2-ethylhexylthio group, a nonylthio group, a decylthio group, a 3,7-dimethyloctylthio group, a laurylthio group and a trifluoromethylthio group. Among these groups, a pentylthio group, a hexylthio group, an octylthio group, a 2-ethylhexylthio group, a decylthio group, and a 3,7-dimethyloctylthio group are preferred from the viewpoint of a balance between solubility of a polymer compound in an organic solvent, photoelectric conversion element characteristics, ease of synthesis of a monomer and the like and heat resistance of a polymer compound.

The aryl group is an atomic group in which one hydrogen atom has been removed from an aromatic hydrocarbon and includes those having a fused ring, and those in which an independent benzene ring, or two or more fused rings is/are bonded directly or bonded via a group such as vinylene or the like. The number of carbon atoms of the aryl group is usually from about 6 to 60, and preferably from about 6 to 30. Examples of the aryl group include a phenyl group, a C₁-C₁₂ alkoxyphenyl group (C₁-C₁₂ denotes that the number of carbon atoms (herein, the number of carbon atoms in an alkoxy group of an alkoxyphenyl group) of an organic group described immediately behind C₁-C₁₂ is from 1 to 12, the same shall apply hereinafter), a C₁-C₁₂ alkylphenyl group, a 1-naphthyl group, a 2-naphthyl group, a 1-anthrathenyl group, a 2-anthrathenyl group, a 9-anthrathenyl group and a pentafluorophenyl group. Among these groups, a C₁-C₁₂ alkoxyphenyl group and a C₁-C₁₂ alkylphenyl group are preferred from the viewpoint of solubility of a polymer compound in an organic solvent, photoelectric conversion element characteristics, ease of synthesis of a monomer and the like. Examples of the C₁-C₁₂ alkoxyphenyl group include a methoxyphenyl group, an ethoxyphenyl group, a propyloxyphenyl group, an i-propyloxyphenyl group, a butoxyphenyl group, an i-butoxyphenyl group, a s-butoxyphenyl group, a t-butoxyphenyl group, a pentyloxyphenyl group, a hexyloxyphenyl group, a cyclohexyloxyphenyl group, a heptyloxyphenyl group, an octyloxyphenyl group, a 2-ethylhexyloxyphenyl group, a nonyloxyphenyl group, a decyloxyphenyl group, a 3,7-dimethyloctyloxyphenyl group and a lauryloxyphenyl group. Furthermore, examples of the C₁-C₁₂ alkylphenyl group include a methylphenyl group, an ethylphenyl group, a dimethylphenyl group, a propylphenyl group, a mesityl group, a methylethylphenyl group, an i-propylphenyl group, a butylphenyl group, an i-butylphenyl group, a s-butylphenyl group, a t-butylphenyl group, a pentylphenyl group, an isoamylphenyl group, a hexylphenyl group, a heptylphenyl group, an octylphenyl group, a nonylphenyl group, a decylphenyl group and a dodecylphenyl group.

The number of carbon atoms of the aryloxy group is usually from about 6 to 60, and preferably from about 6 to 30. Examples of the aryloxy group include a phenoxy group, a C₁-C₁₂ alkoxyphenoxy group, a C₁-C₁₂ alkylphenoxy group, a 1-naphthyloxy group, a 2-naphthyloxy group and a pentafluorophenyloxy group. Among these groups, a C₁-C₁₂ alkoxyphenoxy group and a C₁-C₁₂ alkylphenoxy group are preferred from the viewpoint of solubility of a polymer compound in an organic solvent, photoelectric conversion element characteristics, ease of synthesis of a monomer and the like. Examples of the C₁-C₁₂ alkoxyphenoxy group include a methoxyphenoxy group, an ethoxyphenoxy group, a propyloxyphenoxy group, an i-propyloxyphenoxy group, a butoxyphenoxy group, an i-butoxyphenoxy group, a s-butoxyphenoxy group, a t-butoxyphenoxy group, a pentyloxyphenoxy group, a hexyloxyphenoxy group, a cyclohexyloxyphenoxy group, a heptyloxyphenoxy group, an octyloxyphenoxy group, a 2-ethylhexyloxyphenoxy group, a nonyloxyphenoxy group, a decyloxyphenoxy group, a 3,7-dimethyloctyloxyphenoxy group and a lauryloxyphenoxy group. Furthermore, examples of the C₁-C₁₂ alkylphenoxy group include a methylphenoxy group, an ethylphenoxy group, a dimethylphenoxy group, a propylphenoxy group, a 1,3,5-trimethylphenoxy group, a methylethylphenoxy group, an i-propylphenoxy group, a butylphenoxy group, an i-butylphenoxy group, a s-butylphenoxy group, a t-butylphenoxy group, a pentylphenoxy group, an isoamylphenoxy group, a hexylphenoxy group, a heptylphenoxy group, an octylphenoxy group, a nonylphenoxy group, a decylphenoxy group and a dodecylphenoxy group.

The number of carbon atoms of the arylthio group is usually from about 6 to 60, and preferably from about 6 to 30. Examples of the arylthio group include a phenylthio group, a C₁-C₁₂ alkoxyphenylthio group, a C₁-C₁₂ alkylphenylthio group, a 1-naphthylthio group, a 2-naphthylthio group and a pentafluorophenylthio group. Among these groups, a C₁-C₁₂ alkoxyphenylthio group and a C₁-C₁₂ alkylphenylthio group are preferred from the viewpoint of solubility of a polymer compound in an organic solvent, photoelectric conversion element characteristics, ease of synthesis of a monomer and the like.

The number of carbon atoms of the arylalkyl group is usually from about 7 to 60, and preferably from about 7 to 30. Examples of the arylalkyl group include a phenyl-C₁-C₁₂ alkyl group, a C₁-C₁₂ alkoxyphenyl-C₁-C₁₂ alkyl group, a C₁-C₁₂ alkylphenyl-C₁-C₁₂ alkyl group, a 1-naphthyl-C₁-C₁₂ alkyl group and a 2-naphthyl-C₁-C₁₂ alkyl group. Among these groups, a C₁-C₁₂ alkoxyphenyl-C₁-C₁₂ alkyl group and a C₁-C₁₂ alkylphenyl-C₁-C₁₂ alkyl group are preferred from the viewpoint of solubility of a polymer compound in an organic solvent, photoelectric conversion element characteristics, ease of synthesis of a monomer and the like.

The number of carbon atoms of the arylalkoxy group is usually from about 7 to 60, and preferably from about 7 to 30. Examples of the arylalkoxy group include phenyl-C₁-C₁₂ alkoxy groups such as a phenylmethoxy group, a phenylethoxy group, a phenylbutoxy group, a phenylpentyloxy group, a phenylhexyloxy group, a phenylhepthyloxy group, and a phenyloctyloxy group; a C₁-C₁₂ alkoxyphenyl-C₁-C₁₂ alkoxy group, a C₁-C₁₂ alkylphenyl-C₁-C₁₂ alkoxy group, a 1-naphthyl-C₁-C₁₂ alkoxy group and a 2-naphthyl-C₁-C₁₂ alkoxy group. Among these groups, a C₁-C₁₂ alkoxyphenyl-C₁-C₁₂ alkoxy group and a C₁-C₁₂ alkylphenyl-C₁-C₁₂ alkoxy group are preferred from the viewpoint of solubility of a polymer compound in an organic solvent, photoelectric conversion element characteristics, ease of synthesis of a monomer and the like.

The number of carbon atoms of the arylalkylthio group is usually from about 7 to 60, and preferably from about 7 to 30. Examples of the arylalkylthio group include a phenyl-C₁-C₁₂ alkylthio group, a C₁-C₁₂ alkoxyphenyl-C₁-C₁₂ alkylthio group, a C₁-C₁₂ alkylphenyl-C₁-C₁₂ alkylthio group, a 1-naphthyl-C₁-C₁₂ alkylthio group and a 2-naphthyl-C₁-C₁₂ alkylthio group. Among these groups, a C₁-C₁₂ alkoxyphenyl-C₁-C₁₂ alkylthio group and a C₁-C₁₂ alkylphenyl-C₁-C₁₂ alkylthio group are preferred from the viewpoint of solubility of a polymer compound in an organic solvent, photoelectric conversion element characteristics, ease of synthesis of a monomer and the like.

The number of carbon atoms of the alkenyl group is usually from about 2 to 30, and preferably from about 2 to 15. Examples of the alkenyl group include a vinyl group, a 1-propylenyl group, a 2-propylenyl group, a butenyl group, a pentenyl group, a hexenyl group, a heptenyl group, an octenyl group and a cyclohexenyl group. Further, the alkenyl group includes dienyl and trienyl groups such as a 1,3-butadienyl group, a cyclohexa-1,3-dienyl group, and a 1,3,5-hexatrienyl group.

The number of carbon atoms of the alkynyl group is usually from about 2 to 30, and preferably from about 2 to 15.

Examples of the alkynyl group include an ethynyl group, a 1-propynyl group, a 2-propylenyl group, a butynyl group, a pentynyl group, a hexynyl group, a heptynyl group, an octynyl group and a cyclohexylethynyl group. Further, the alkynyl group includes diynyl groups such as a 1,3-butadiynyl group.

The substituted amino group includes an amino group substituted with at least one group selected from the group consisting of an alkyl group, an aryl group, an arylalkyl group, and a monovalent heterocyclic group.

The alkyl group, aryl group, arylalkyl group, and monovalent heterocyclic group included in the substituted amino group may have a substituent. The number of carbon atoms of the substituted amino group is usually from about 2 to 60, and preferably from about 2 to 30, excluding the number of carbon atoms of the substituent which may be included in the alkyl group or the like. Examples of the substituted amino group include a dimethylamino group, a diethylamino group, a dipropylamino group, a diisopropylamino group, a dibutylamino group, a diisobutylamino group, a di-t-butylamino group, a dipentylamino group, a dihexylamino group, a dicyclohexylamino group, a diheptylamino group, a dioctylamino group, a di-2-ethylhexylamino group, a dinonylamino group, a didecylamino group, a di-3,7-dimethyloctylamino group; a dilaurylamino group, a dicyclopentylamino group, a dicyclohexylamino group, a pyrrolidyl group, a piperidyl group, a ditrifluoromethylamino group, a phenylamino group, a diphenylamino group, a di(C₁-C₁₂ alkoxyphenyl)amino group, a di(C₁-C₁₂ alkylphenyl)amino group, a di-1-naphthylamino group, a di-2-naphthylamino group, a dipentafluorophenylamino group, a dipyridylamino group, a dipyridazinylamino group, a dipyrimidylamino group, a dipyrazylamino group, a ditriazylamino group, a di(phenyl-C₁-C₁₂ alkylamino group, di(C₁-C₁₂ alkoxyphenyl-C₁-C₁₂ alkyl)amino group and a di(C₁-C₁₂ alkylphenyl-C₁-C₁₂ alkyl)amino group.

The substituted silyl group includes a silyl group substituted with at least one group selected from the group consisting of an alkyl group, an aryl group, an arylalkyl group and a monovalent heterocyclic group.

The alkyl group, aryl group, arylalkyl group and monovalent heterocyclic group included in the substituted silyl group may have a substituent. The number of carbon atoms of the substituted silyl group is usually from about 3 to 90, and preferably from about 3 to 45, excluding the number of carbon atoms of the substituent which may be included in the alkyl group or the like. Examples of the substituted silyl group include a trimethylsilyl group, a triethylsilyl group, a tripropylsilyl group, a tri-i-propylsilyl group, a dimethyl-i-propylsilyl group, a diethyl-i-propylsilyl group, a t-butylsilyldimethylsilyl group, a pentyldimethylsilyl group, a hexyldimethylsilyl group, a heptyldimethylsilyl group, an octyldimethylsilyl group, a 2-ethylhexyl-dimethylsilyl group, a nonyldimethylsilyl group, a decyldimethylsilyl group, a 3,7-dimethyloctyl-dimethylsilyl group, a lauryldimethylsilyl group, a phenyl-C₁-C₁₂ alkylsilyl group, a C₁-C₁₂ alkoxyphenyl-C₁-C₁₂ alkylsilyl group, a C₁-C₁₂ alkylphenyl-C₁-C₁₂ alkylsilyl group, a 1-naphthyl-C₁-C₁₂ alkylsilyl group, a 2-naphthyl-C₁-C₁₂ alkylsilyl group, a phenyl-C₁-C₁₂ alkyldimethylsilyl group, a triphenylsilyl group, a tri-p-xylylsilyl group, a tribenzylsilyl group, a diphenylmethylsilyl group, a t-butyldiphenylsilyl group and a dimethylphenylsilyl group.

The number of carbon atoms of the acyl group is usually from about 2 to 30, and preferably from about 2 to 15. Examples of the acyl group include an acetyl group, a propionyl group, a butyryl group, an isobutyryl group, a pivaloyl group, a benzoyl group, a trifluoroacetyl group and a pentafluorobenzoyl group.

The number of carbon atoms of the acyloxy group is usually from about 2 to 30, and preferably from about 2 to 15. Examples of the acyloxy group include an acetoxy group, a propionyloxy group, a butyryloxy group, an isobutyryloxy group, a pivaloyloxy group, a benzoyloxy group, a trifluoroacetyloxy group and a pentafluorobenzoyloxy group.

The number of carbon atoms of the imine residue is from about 2 to 30, and preferably from about 2 to 15. Examples of the imine residue include groups represented by the structural formulas shown below and the like. In the structural formula shown below, a wavy line represents a bond and means that geometrical isomers such as a cis-isomer and a trans-isomer are sometimes included depending on the kind of the imine residue.

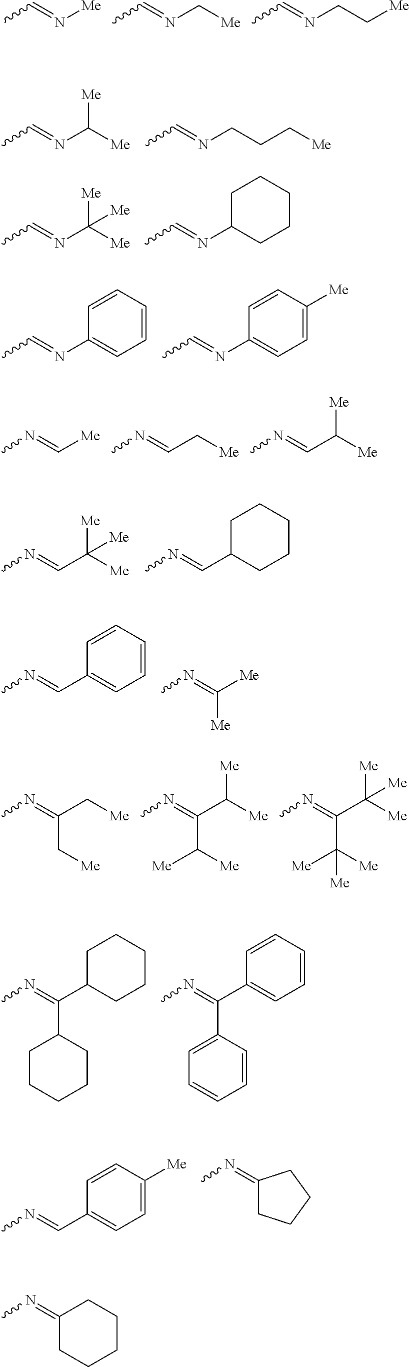

The number of carbon atoms of the amide group is usually from about 2 to 30, and preferably from about 2 to 15. Examples of the amide group include a formamide group, an acetamide group, a propionamide group, a butyroamide group, a benzamide group, a trifluoroacetamide group, a pentafluorobenzamide group, a diformamide group, a diacetamide group, a dipropionamide group, a dibutyroamide group, a dibenzamide group, a ditrifluoroacetamide group and a dipentafluorobenzamide group.

The acid imide group includes a residue obtained by removing hydrogen atoms bonded to nitrogen atoms of an acid imide from the acid imide. The number of carbon atoms of the acid imide group is usually from about 4 to 30, and preferably from about 4 to 15. Examples of the acid imide group include groups represented by the structural formulas shown below.

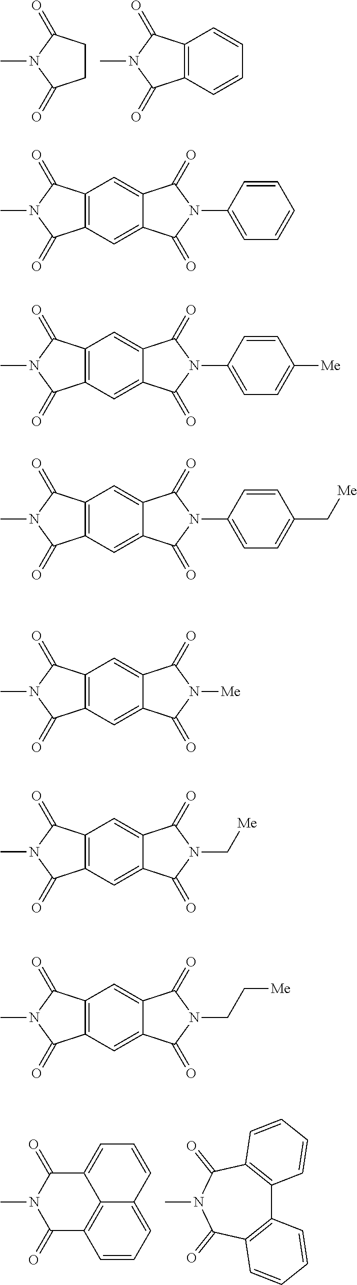

The monovalent heterocyclic group refers to an atomic group remaining after removing one hydrogen atom from a heterocyclic compound.

The number of carbon atoms of the monovalent heterocyclic group is usually from about 2 to 30, and preferably from about 2 to 15.

In the monovalent heterocyclic group, a heterocycle may have a substituent. However, the number of carbon atoms of the substituent on the heterocycle is not included in the number of carbon atoms described above. Herein, the heterocyclic compounds refer to those in which elements constituting a ring are not only carbon atoms, but also hetero atoms such as oxygen, sulfur, nitrogen, phosphorus and boron contained in the ring, among organic compounds having a cyclic structure. Examples of the monovalent heterocyclic group include a thienyl group, a C₁-C₁₂ alkylthienyl group, a pyrrolyl group, a furyl group, a pyridyl group, a C₁-C₁₂ alkylpyridyl group, a piperidyl group, a quinolyl group and an isoquinolyl group. Among these groups, a monovalent aromatic heterocyclic group is preferred, and a thienyl group, a C₁-C₁₂ alkylthienyl group, a pyridyl group, and a C₁-C₁₂ alkylpyridyl group are particularly preferred.

The substituted carboxyl group includes a carboxyl group substituted with an alkyl group, an aryl group, an arylalkyl group or a monovalent heterocyclic group. The alkyl group, aryl group, arylalkyl group, and monovalent heterocyclic group included in the substituted carboxyl group may have a substituent. The number of carbon atoms of the substituted carboxyl group is usually from about 2 to 30, and preferably from about 2 to 15, excluding the number of carbon atoms of the substituent which may be included in the alkyl group or the like. Examples of the substituted carboxyl group include a methoxycarbonyl group, an ethoxycarbonyl group, a propoxycarbonyl group, an i-propoxycarbonyl group, a butoxycarbonyl group, an i-butoxycarbonyl group, a s-butoxycarbonyl group, a t-butoxycarbonyl group, a pentyloxycarbonyl group, a hexyloxycarbonyl group, a cyclohexyloxycarbonyl group, a heptyloxycarbonyl group, an octyloxycarbonyl group, a 2-ethylhexyloxycarbonyl group, a nonyloxycarbonyl group, a decyloxycarbonyl group, a 3,7-dimethyloctyloxycarbonyl group, a dodecyloxycarbonyl group, a trifluoromethoxycarbonyl group, a pentafluoroethoxycarbonyl group, a perfluorobutoxycarbonyl group, a perfluorohexyloxycarbonyl group, a perfluorooctyloxycarbonyl group, a phenoxycarbonyl group, a naphthoxycarbonyl group and a pyridyloxycarbonyl group.

The number of carbon atoms of the heteroaryloxy group (a group represented by Q¹-O—, Q¹ representing a monovalent heterocyclic group) is usually from about 2 to 30, and preferably from about 2 to 15. In the heteroaryloxy group, the monovalent heterocyclic group may have a substituent. However, the number of carbon atoms of the substituent on the monovalent heterocyclic group is not included in the number of carbon atoms of the heteroaryloxy group described above. Examples of the heteroaryloxy group include a thienyloxy group, C₁-C₁₂ alkylthienyloxy group, a pyrrolyloxy group, a furyloxy group, a pyridyloxy group, a C₁-C₁₂ alkylpyridyloxy group, an imidazolyloxy group, a pyrazolyloxy group, a triazolyloxy group, an oxazolyloxy group, a thiazoleoxy group and a thiadiazoleoxy group. Q¹ is preferably a monovalent aromatic heterocyclic group.

The number of carbon atoms of the heteroarylthio group (a group represented by Q²-S—, Q² representing a monovalent heterocyclic group) is usually from about 2 to 30, and preferably from about 2 to 15. In the heteroarylthio group, the monovalent heterocyclic group may have a substituent. However, the number of carbon atoms of the substituent on the monovalent heterocyclic group is not included in the number of carbon atoms of the heteroarylthio group described above. Examples of the heteroarylthio group include a thienylmercapto group, a C₁-C₁₂ alkylthienylmercapto group, a pyrrolylmercapto group, a furylmercapto group, a pyridylmercapto group, a C₁-C₁₂ alkylpyridylmercapto group, an imidazolylmercapto group, a pyrazolylmercapto group, a triazolylmercapto group, an oxazolylmercapto group, a thiazolemercapto group and a thiadiazolemercapto group. Further, Q² is preferably a monovalent aromatic heterocyclic group.

In the formula (1), R¹ and R⁴ are the same or different and each represent a monovalent group.

Examples of the monovalent group include an alkyl group, an aryl group, an arylalkyl group, an alkenyl group, an alkynyl group, a substituted silyl group, an acyl group, a monovalent heterocyclic group and a substituted carboxyl group.

Examples of these alkyl group, aryl group, arylalkyl group, alkenyl group, alkynyl group, substituted silyl group, acyl group, monovalent heterocyclic group, and substituted carboxyl group include the same groups as those shown as examples of the substituent which may be included in ring A, ring B and ring C.

From the viewpoint of stability of a polymer compound, R¹ and R⁴ are preferably alkyl groups; aryl groups, arylalkyl groups or monovalent heterocyclic groups, and more preferably aryl groups.

In the formula (1), R², R³, R⁵ and R⁶ are the same or different and each represent a hydrogen atom or a monovalent group. From the viewpoint of stability of a power generation element, R², R³, R⁵ and R⁶ are preferably monovalent group, and more preferably alkyl groups, aryl groups, arylalkyl groups, alkenyl groups, and alkynyl groups.

The alkyl group represented by R², R³, R⁵ and R⁶ may be linear or branched, and may also be a cycloalkyl group but has no substituent. The number of carbon atoms of the alkyl group is usually from about 1 to 30, and preferably from about 3 to 15 from the viewpoint of solubility of a polymer compound in a solvent.

The hydrogen atom in the alkyl group may be substituted with a fluorine atom. Examples of the alkyl group include a methyl group, an ethyl group, a propyl group, an i-propyl group, a butyl group, an i-butyl group, a s-butyl group, a t-butyl group, a pentyl group, an isoamyl group, a hexyl group, a cyclohexyl group, a heptyl group, an octyl group, a 2-ethylhexyl group, a nonyl group, a decyl group, a 3,7-dimethyloctyl group, a lauryl group, a trifluoromethyl group, a pentafluoroethyl group, a perfluorobutyl group, a perfluorohexyl group and a perfluorooctyl group. Among these groups, a pentyl group, an isoamyl group, a hexyl group, an octyl group, a 2-ethylhexyl group, a decyl group, and a 3,7-dimethyloctyl group are preferred from the viewpoint of a balance between solubility of a polymer compound in an organic solvent, photoelectric conversion element characteristics, ease of synthesis of a monomer and heat resistance of a polymer compound.

Examples of the aryl group, arylalkyl group, alkenyl group, and alkynyl group represented by R², R³, R⁵ and R⁶ include the same groups as those shown as examples of the substituent which may be included in ring A, ring B and ring C.

From the viewpoint of ease of synthesis of a compound, R², R³, R⁵ and R⁶ are preferably alkyl groups, aryl groups or arylalkyl groups.

From the viewpoint of ease of synthesis of a compound having a structure represented by the formula (1), the structure represented by the formula (1) is preferably C2-symmetric.

Further, from the viewpoint of heat resistance of a polymer compound and film-forming properties, R², R³, R⁵ and R⁶ in the formula (1) are preferably groups represented by the formula (2):

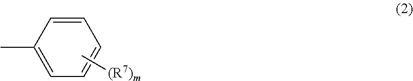

wherein R⁷ represents a halogen atom, an alkyl group, an alkoxy group, an alkylthio group, an aryl group, an aryloxy group, an arylthio group, an arylalkyl group, an arylalkyloxy group, an arylalkylthio group, an alkenyl group, an alkynyl group, a substituted amino group, or a substituted silyl group, and m represents an integer of from 0 to 5. When m is 2 or more, plural R⁷(s) may be the same or different from each other.

Examples of the halogen atom, alkyl group, alkoxy group, alkylthio group, aryl group, aryloxy group, arylthio group, arylalkyl group, arylalkyloxy group, arylalkylthio group, alkenyl group, alkynyl group, substituted amino group, and substituted silyl group represented by R⁷ include the same groups as those shown as examples of the substituent which may be included in ring A, ring B and ring C. Among these groups, an alkyl group, an alkoxy group, an aryl group, an aryloxy group, and a substituted amino group are preferred from the viewpoint of stability of a polymer compound.

From the viewpoint of stability of a photoelectric conversion element, m is preferably 0.

From the viewpoint of stability of a polymer compound, R⁷ is preferably an alkyl group having 3 or more carbon atoms, an alkoxy group having 3 or more carbon atoms, an alkylthio group having 3 or more carbon atoms, an alkenyl group having 3 or more carbon atoms, an alkynyl group having 3 or more carbon atoms, or a substituted amino group having 3 or more carbon atoms.

From the viewpoint of conductivity of a polymer compound, it is preferred to polymerize at the bonding position where conjugates are connected. Among these, regarding the aromatic amine residue, the group in which at least one hydrogen atom has been removed from a structure represented by the formula (1) is preferably a group represented by the formula (3):

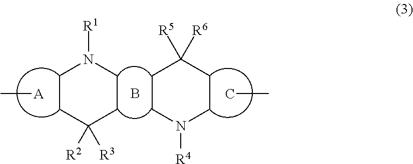

wherein ring A, ring B, ring C, R¹, R², R³, R⁴, R⁵ and R⁶ have the same meanings as defined above.

In the group represented by the formula (3), ring A and ring C in the formula (3) are preferably benzene rings from the viewpoint of ease of synthesis of a compound. It is more preferred that the polymer compound used in the present invention contains a repeating unit represented by the formula (4):

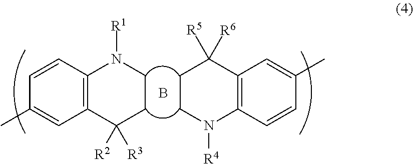

wherein ring B, R¹, R⁴, R², R³, R⁵ and R⁶ have the same meanings as defined above.

Examples of the repeating unit represented by the formula (4) include the repeating units shown below.

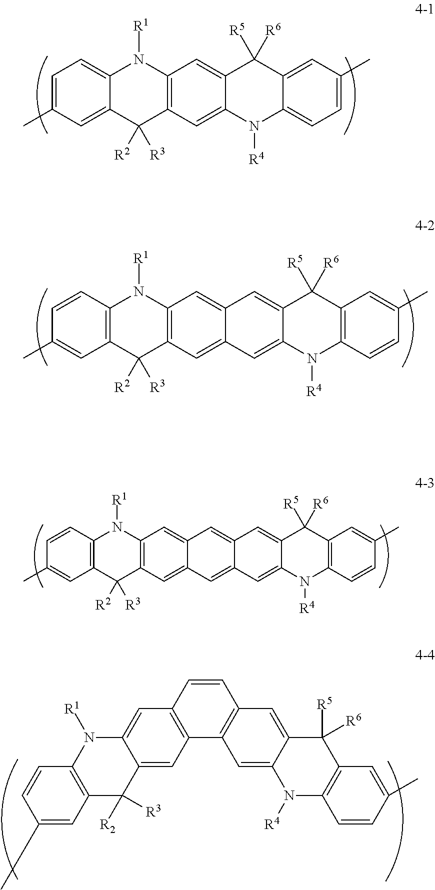

In the formula (4), ring B is preferably an aromatic hydrocarbon ring, more preferably a benzene ring, a naphthalene ring, an anthracene ring or a phenanthrene ring, and still more preferably a benzene ring, from the viewpoint of heat resistance of a polymer compound, photoelectric conversion element characteristics and the like.

The polymer compound used in the present invention may have at least one group selected from the group consisting of a group represented by the formula (5-1) and a group represented by the formula (5-2):

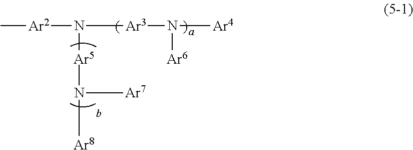

wherein Ar², Ar³, Ar⁴ and Ar⁵ are the same or different and each represent an arylene group or a divalent heterocyclic group, Ar⁶, Ar⁷ and Ar⁸ are the same or different and each represent an aryl group or a monovalent heterocyclic group, a and b are the same or different and represent 0 or a positive integer, when plural Ar³(s) are present, they may be respectively the same or different, when plural Ar⁵(s) are present, they may be respectively the same or different, when plural Ar⁶(s) are present, they may be respectively the same or different, and when plural Ar⁷(s) are present, they may be respectively the same or different; and

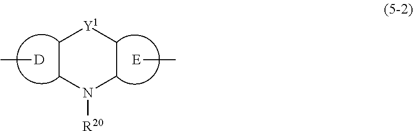

wherein ring D and ring E are the same or different and each represent an aromatic ring having a bond, Y¹ represents —O—, —S—, or —C(═O)—, and R²⁰ represents a monovalent group.

Examples of the aryl group and monovalent heterocyclic group represented by Ar⁶ to Ar⁸ include the same groups as those shown as examples of the substituent which may be included in ring A, ring B and ring C.

The number of carbon atoms of the arylene group represented by Ar² to Ar⁵ is usually from about 6 to 60, and preferably from 6 to 20. Examples of the arylene group include phenylene groups (for example, the general formulas 1 to 3 shown below), naphthalenediyl groups (for example, the general formulas 4 to 13 shown below), anthrathenylene groups (for example, the general formulas 14 to 19 shown below), biphenylene groups (for example, the general formulas 20 to 25 shown below), triphenylene groups (for example, the general formulas 26 to 28 shown below) and fused ring compound groups (for example, the general formulas 29 to 38 shown below). R(s) in the formulas shown below may be the same or different and each represent a hydrogen atom, a halogen atom, an alkyl group, an alkoxy group, an alkylthio group, an aryl group, an aryloxy group, an arylthio group, an arylalkyl group, an arylalkyloxy group, an arylalkylthio group, an alkenyl group, an alkynyl group, a heteroaryloxy group, or a heteroarylthio group. Examples of the halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group, or heteroarylthio group represented by R include the same groups as those shown as examples of the substituent which may be included in ring A, ring B and ring C. The number of carbon atoms of the substituent R is not included in the number of carbon atoms of the arylene group described above.

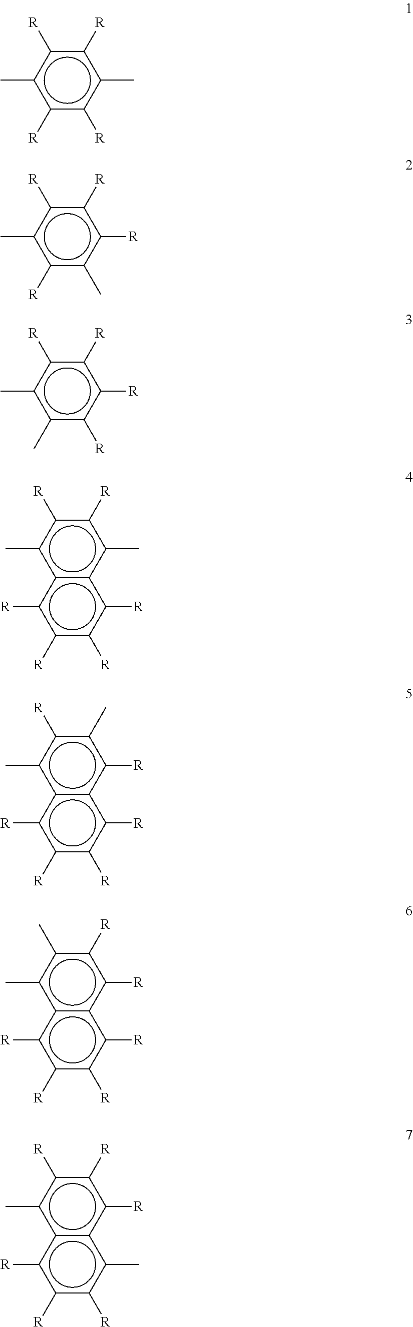

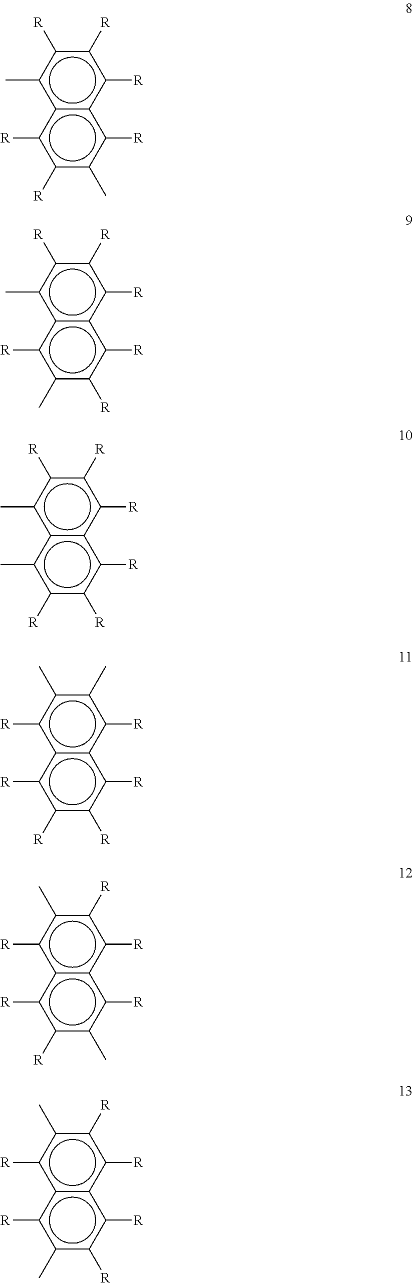

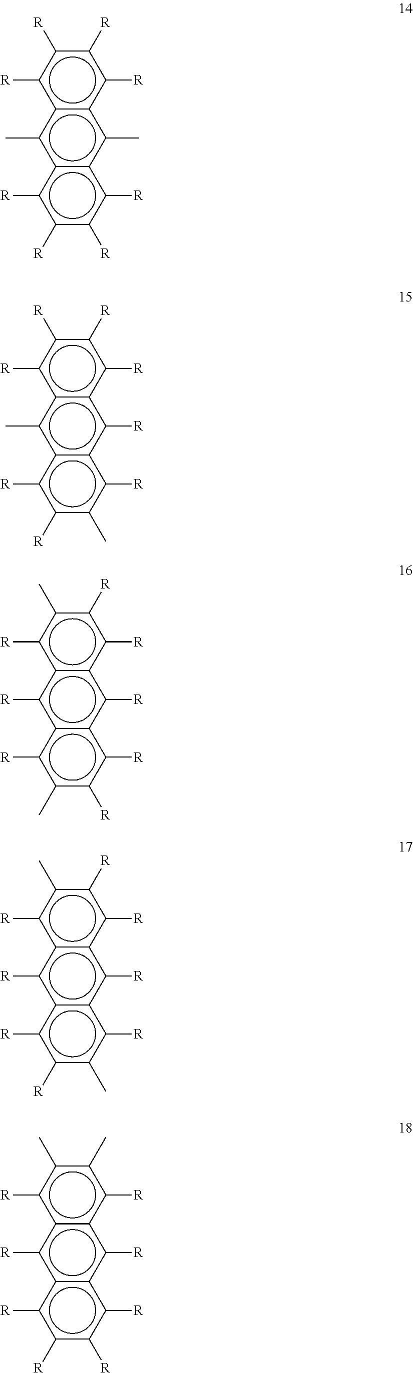

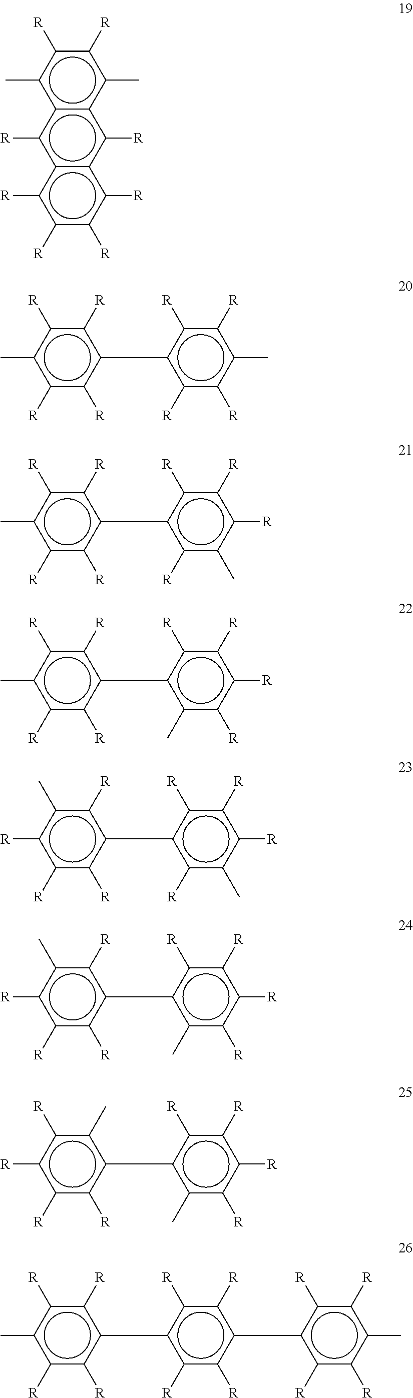

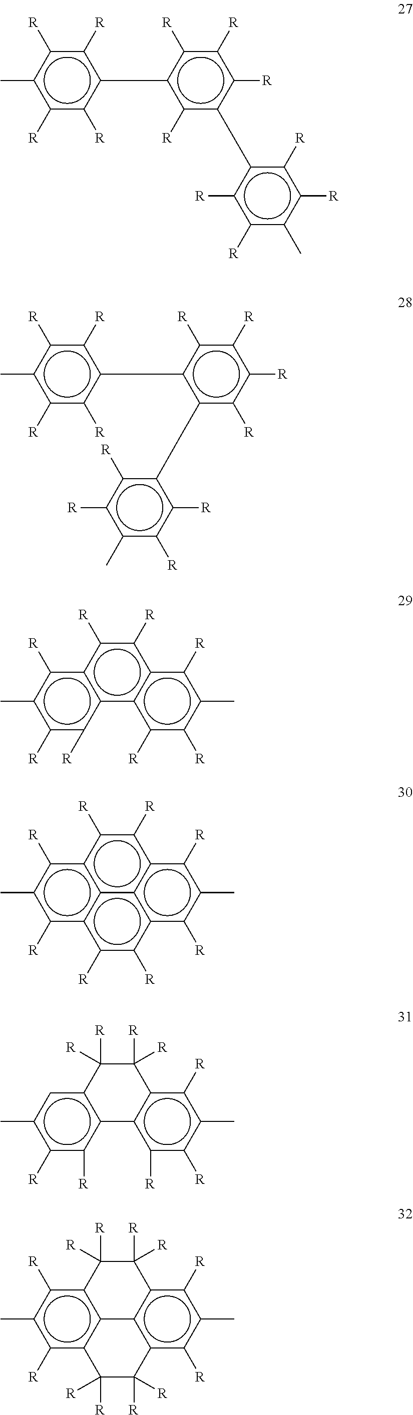

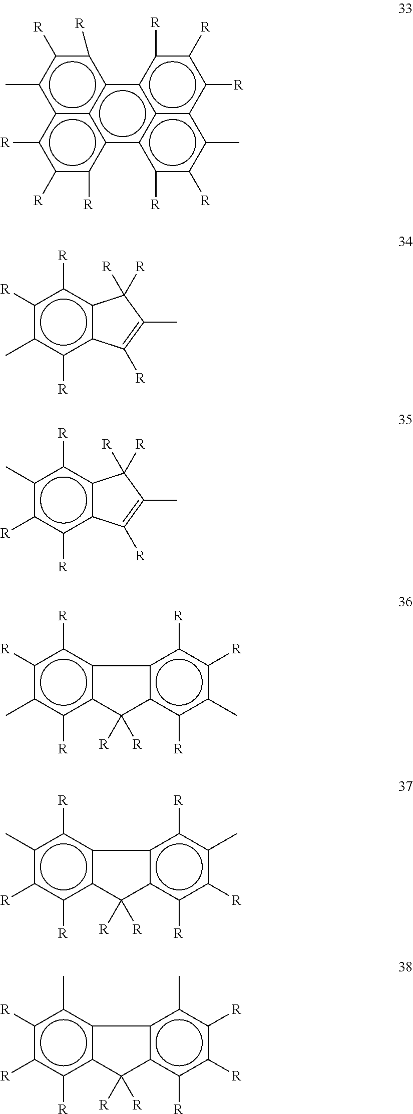

The divalent heterocyclic group represented by Ar² to Ar⁵ refers to an atomic group remaining after removing two hydrogen atoms from a heterocyclic compound. The number of carbon atoms of such a divalent heterocyclic group is usually from about 4 to 60, and preferably from 4 to 20. Herein, the heterocyclic compounds refer to those in which elements constituting a ring are not only carbon atoms, but also hetero atoms such as oxygen, sulfur, nitrogen, phosphorus and boron contained in the ring, among organic compounds having a cyclic structure.

Examples of the divalent heterocyclic group include the groups shown below and the like. In the formulas shown below, R(s) may be the same or different and each represent a hydrogen atom, a halogen atom, an alkyl group, an alkoxy group, an alkylthio group, an aryl group, an aryloxy group, an arylthio group, an arylalkyl group, an arylalkyloxy group, an arylalkylthio group, an alkenyl group, an alkynyl group, a heteroaryloxy group, or a heteroarylthio group. Examples of the halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group, or heteroarylthio group represented by R include the same groups as those shown as examples of the substituent which may be included in ring A, ring B and ring C. The number of carbon atoms of the substituent R is not included in the number of carbon atoms of the divalent heterocyclic group described above.

Examples of the divalent heterocyclic group containing nitrogen as a hetero atom include pyridine-diyl groups (for example, the general formulas 39 to 44 shown below), diazaphenylene groups (for example, the general formulas 45 to 48 shown below), quinolinediyl groups (for example, the general formulas 49 to 63 shown below), quinoxalinediyl groups (for example, the general formulas 64 to 68 shown below), acridinediyl groups (for example, the general formulas 69 to 72 shown below), bipyridyldiyl groups (for example, the general formulas 73 to 75 shown below) and phenanthrolinediyl groups (for example, the general formulas 76 to 78 shown below).

Examples of a group, which contains silicon, nitrogen, sulfur, selenium or the like as a hetero atom and has a fluorene structure, include groups represented by the general formulas 79 to 98 shown below and the like.

Examples of a 5-membered ring heterocyclic group, which contains silicon, nitrogen, sulfur, selenium or the like as a hetero atom, include groups represented by the general formulas 94 to 98 shown below and the like.

Examples of a 5-membered ring fused heterocyclic group, which contains silicon, nitrogen, sulfur, selenium or the like as a hetero atom, include groups represented by the general formulas 99 to 109 shown below, a benzothiadiazole-4,7-diyl group, benzooxadiazole-4,7-diyl group and the like.

Examples of a group, which is a 5-membered ring heterocyclic group containing silicon, nitrogen, sulfur, selenium or the like as a hetero atom and is bonded at the α-position of the hetero atom to form a dimer or an oligomer, include groups represented by the general formulas 110 to 118 shown below and the like.

Examples of a group, which is a 5-membered ring heterocyclic group containing silicon, nitrogen, sulfur, selenium or the like as a hetero atom and is bonded to a phenyl group at the α-position of the hetero atom, include groups represented by the general formulas 112 to 118 shown below and the like.

Examples of a tricyclic group in which a fused heterocyclic group containing nitrogen, oxygen, sulfur or the like as a hetero atom is bonded with a benzene ring or a monocyclic heterocyclic group include groups represented by the general formulas 120 to 125 shown below and the like.

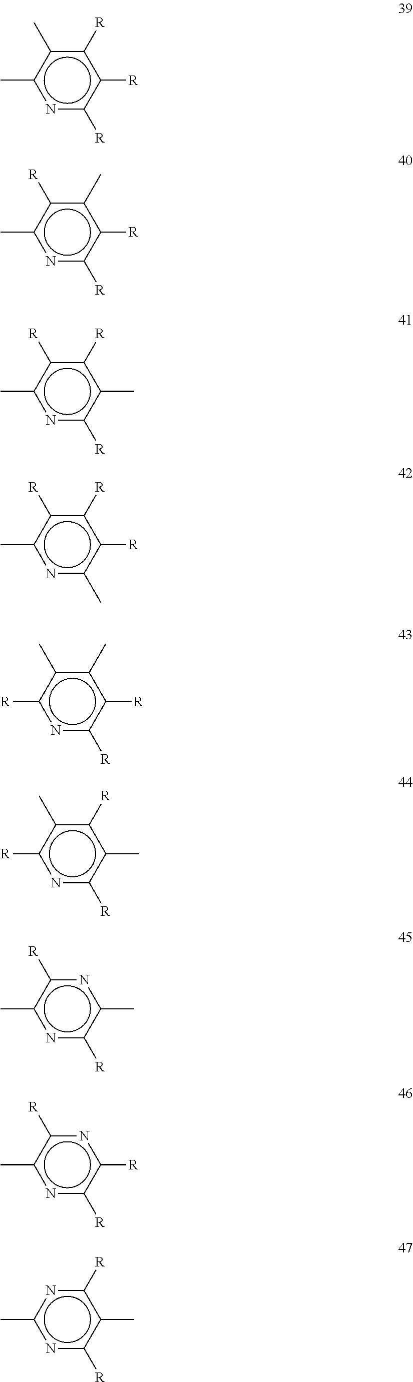

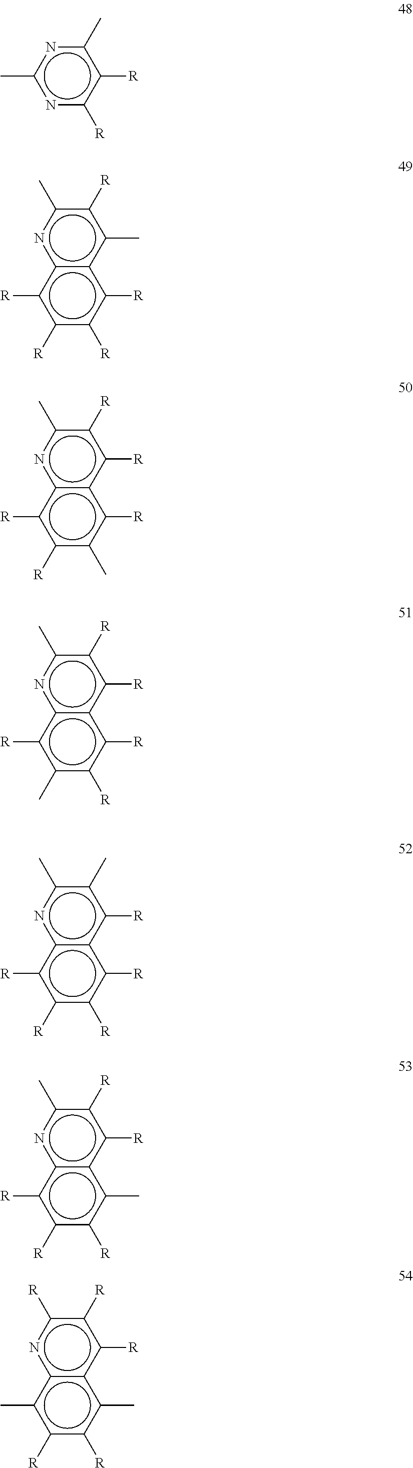

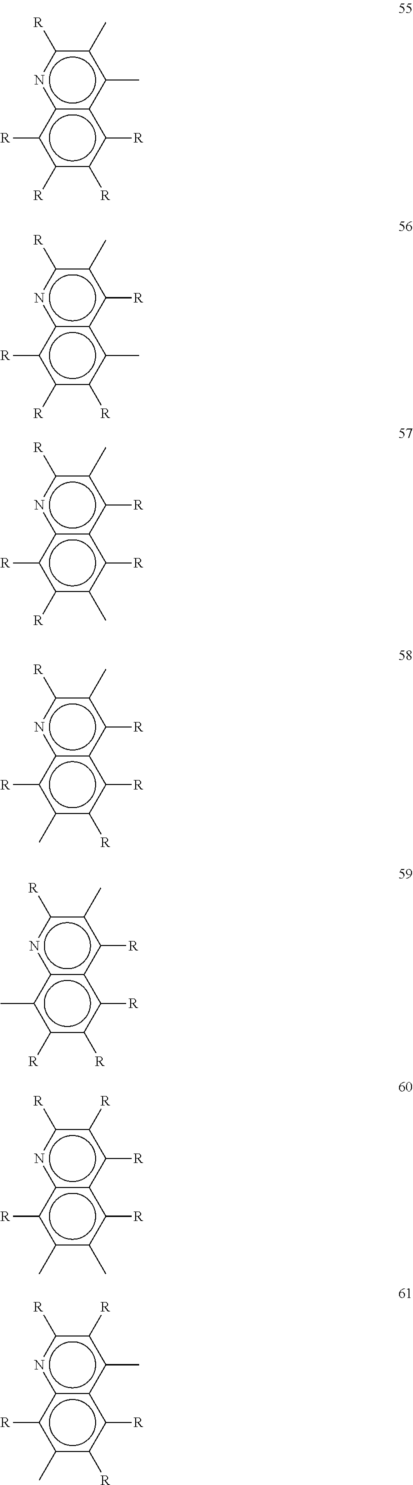

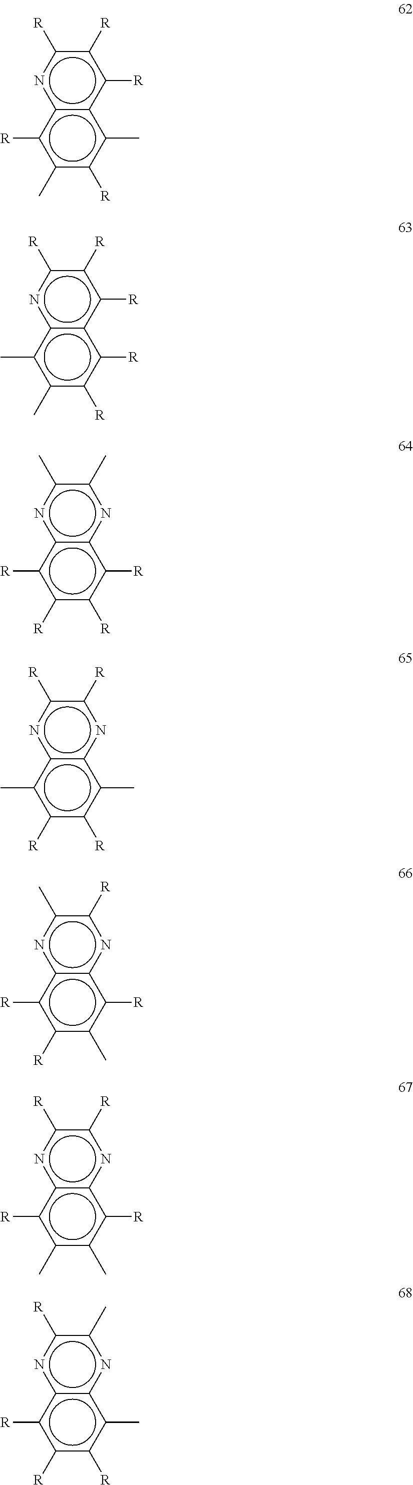

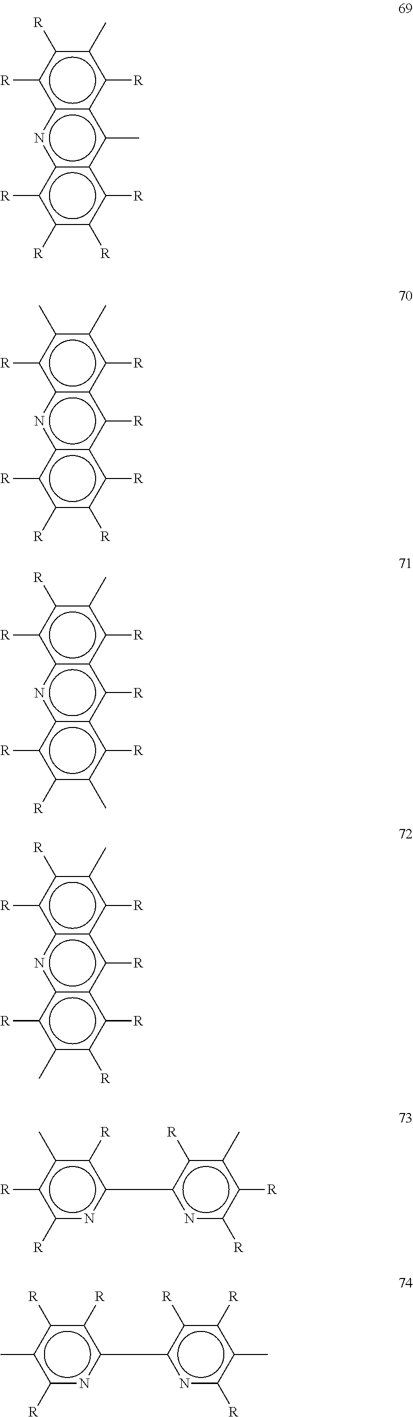

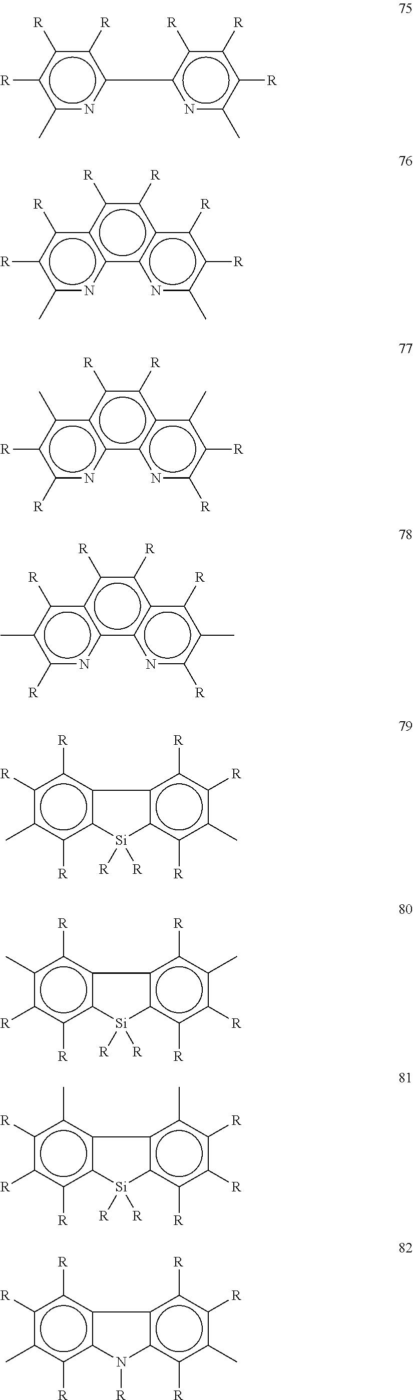

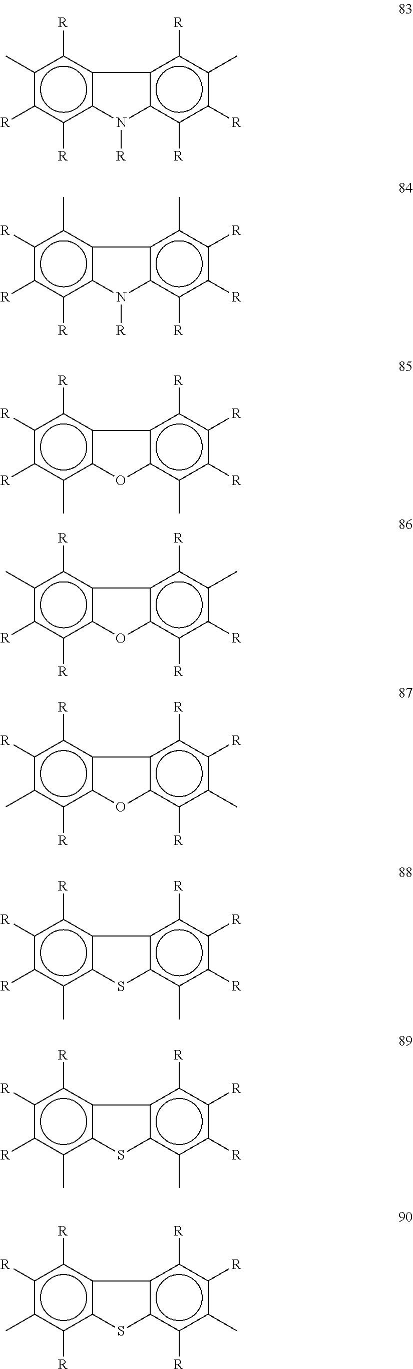

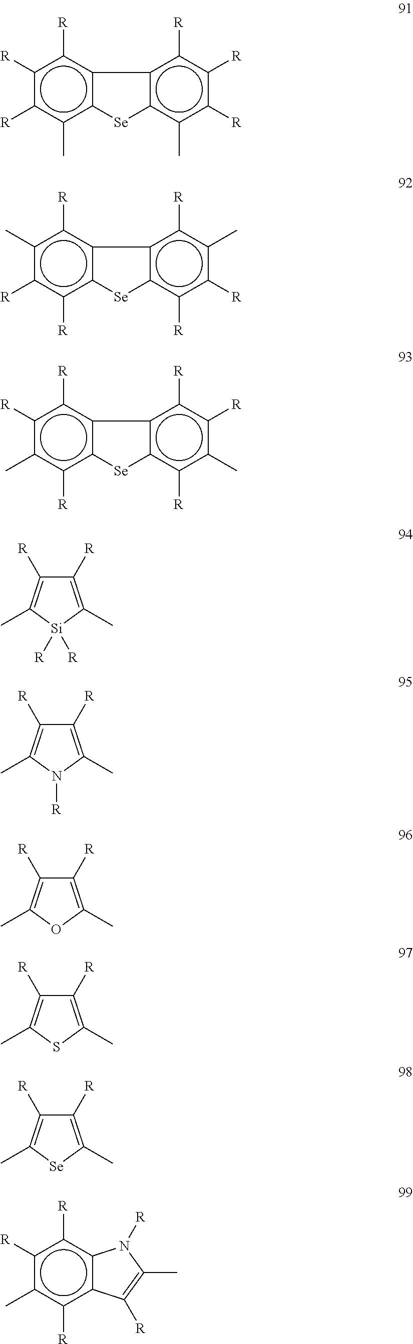

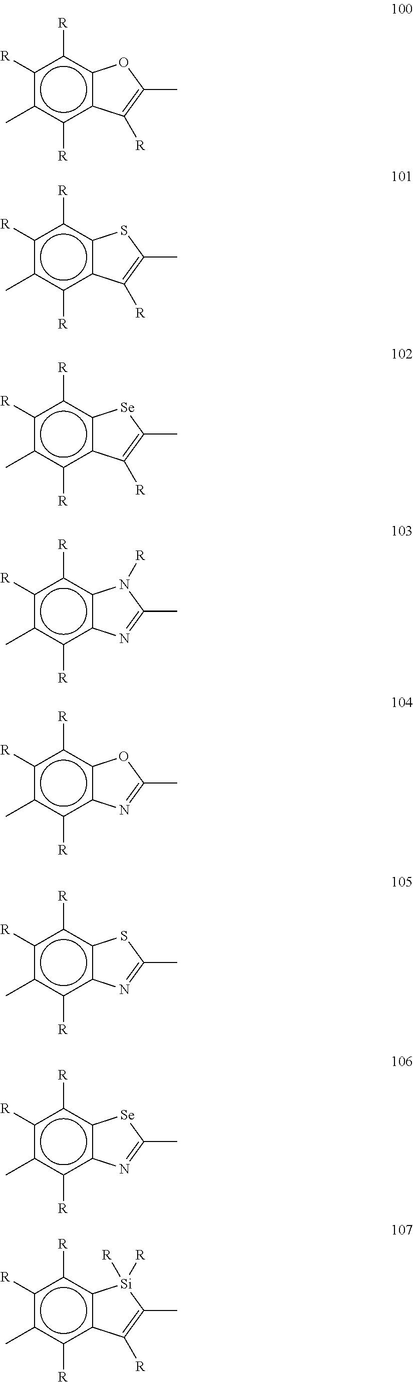

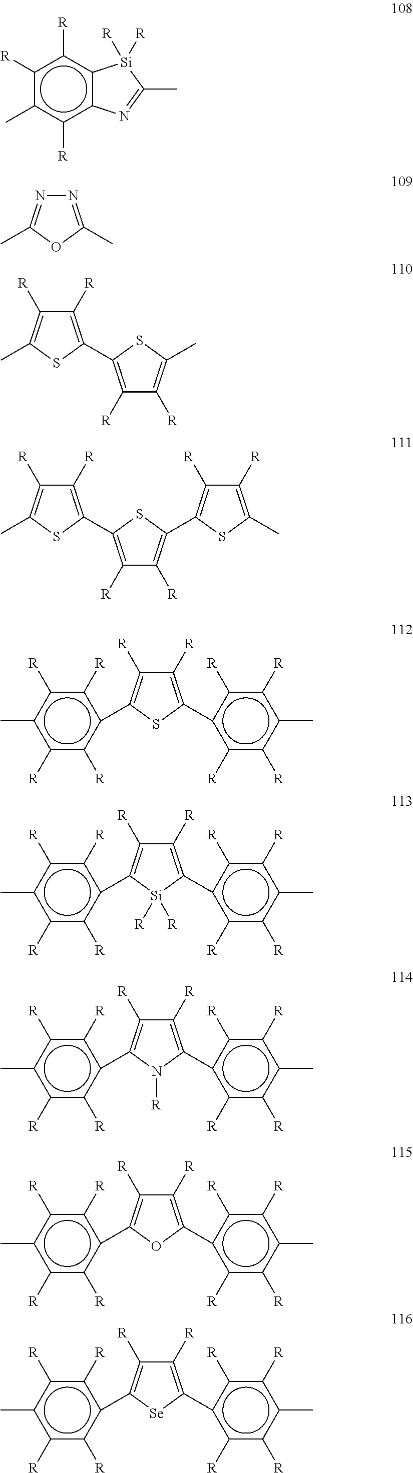

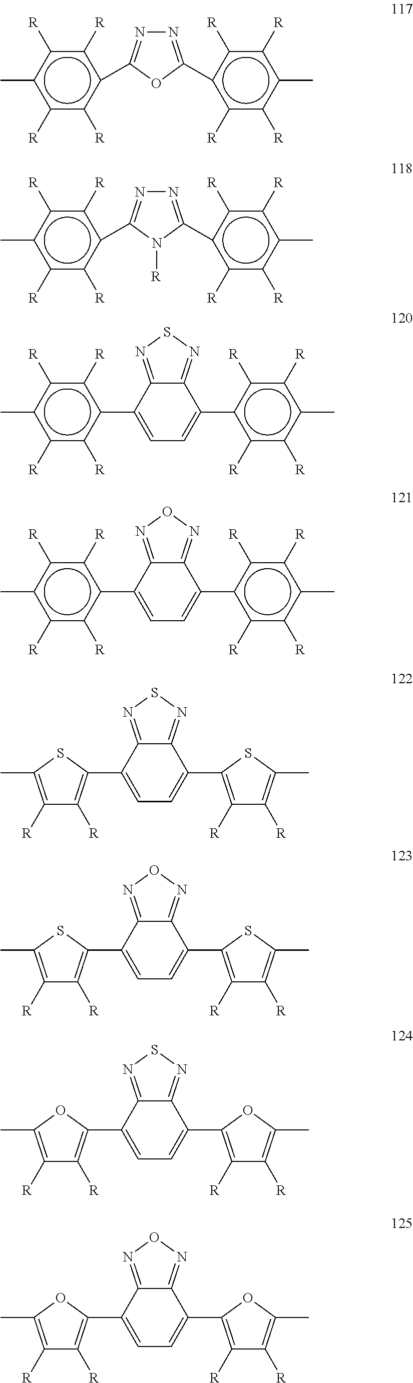

Examples of the group represented by the formula (5-1) include groups represented by the general formulas 133 to 140 shown below and the like.

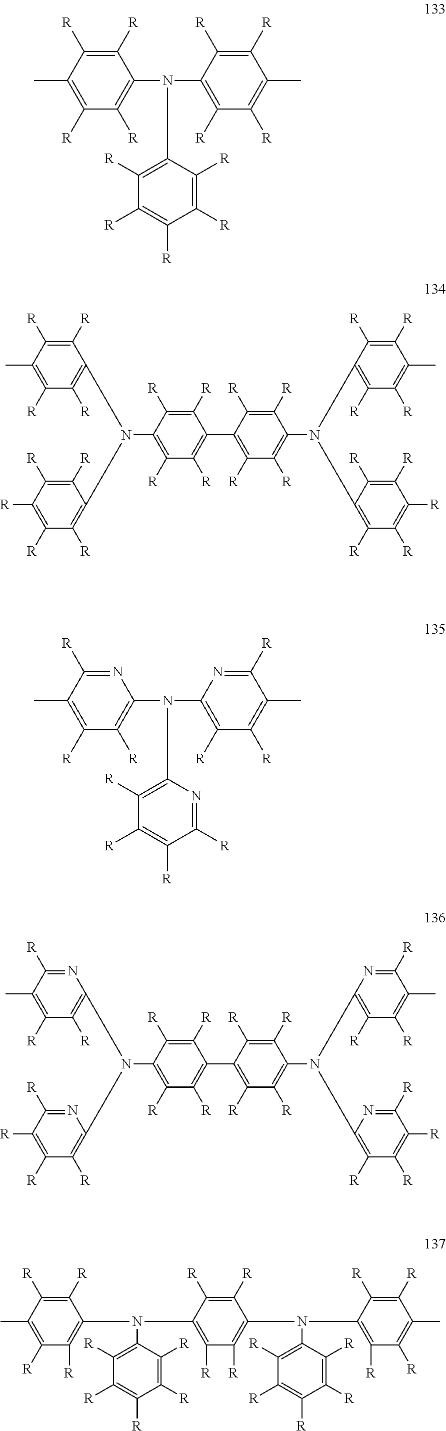

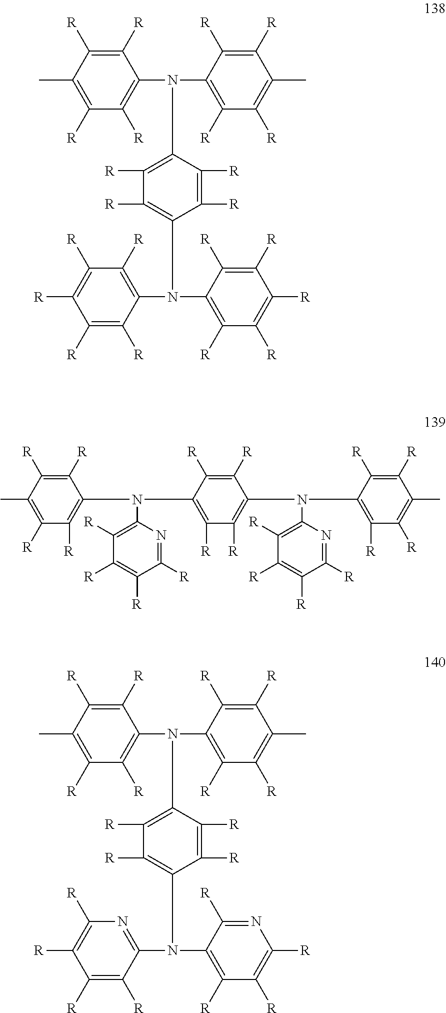

In the general formulas shown above, R(s) may be the same or different and each represent a hydrogen atom, a halogen atom, an alkyl group, an alkoxy group, an alkylthio group, an aryl group, an aryloxy group, an arylthio group, an arylalkyl group, an arylalkyloxy group, an arylalkylthio group, an alkenyl group, an alkynyl group, a heteroaryloxy group, or a heteroarylthio group. Examples of the halogen atom, alkyl group, alkoxy group, alkylthio group, aryl group, aryloxy group, arylthio group, arylalkyl group, arylalkyloxy group, arylalkylthio group, alkenyl group, alkynyl group, heteroaryloxy group, or heteroarylthio group represented by R include the same groups as those shown as examples of the substituent which may be included in ring A, ring B and ring C.

In the group represented by the formula (5-1), it is preferred that Ar², Ar³, Ar⁴ and Ar⁵ are arylene groups, and Ar⁶, Ar⁷ and Ar⁸ are aryl groups, from the viewpoint of element lifetime and element characteristics. Ar², Ar³, Ar⁴ and Ar⁵ are preferably the same or different and each represent a non-substituted phenylene group, a non-substituted biphenylene group, a non-substituted naphthylene group, or a non-substituted anthracenediyl group. Furthermore, from the viewpoint of solubility of a polymer compound in an organic solvent, photoelectric conversion element characteristics and the like, Ar⁶, Ar⁷ and Ar⁸ are preferably aryl groups having 1 or more substituents, more preferably aryl groups having 3 or more substituents, still more preferably phenyl groups having 3 or more substituents, naphthyl groups having 3 or more substituent, or anthrathenyl groups having 3 or more substituents, and further preferably phenyl groups having 3 or more substituents.

Among the group represented by the formula (5-1), a group in which Ar⁶, Ar⁷ and Ar⁸ are groups represented by the formula (6-4) and a+b S 3 is preferred, a group in which a+b=1 is more preferred, and a group in which a=1 and b=0 is still more preferred.

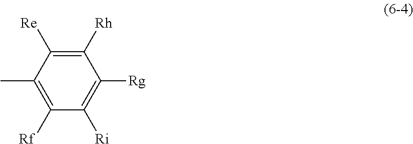

In the formula (6-4), Re, Rf and Rg are the same or different and each represent an alkyl group, an alkoxy group, an alkylthio group, an aryl group, an aryloxy group, an arylthio group, an arylalkyl group, an arylalkoxy group, an arylalkylthio group, an arylalkenyl group, an arylalkynyl group, an amino group, a substituted amino group, a silyl group, a substituted silyl group, a silyloxy group, a substituted silyloxy group, a monovalent heterocyclic group, or a halogen atom. The hydrogen atoms included in Re, Rf and Rg may be substituted with fluorine atoms. Furthermore, Rh and Ri are the same or different and each represent a hydrogen atom, an alkyl group, an alkoxy group, an alkylthio group, an aryl group, an aryloxy group, an arylthio group, an arylalkyl group, an arylalkoxy group, an arylalkylthio group, an arylalkenyl group, an arylalkynyl group, an amino group, a substituted amino group, a silyl group, a substituted silyl group, a silyloxy group, a substituted silyloxy group, a monovalent heterocyclic group, or a halogen atom. The hydrogen atoms included in Rh and Ri may be substituted with fluorine atoms.

Examples of the alkyl group, alkoxy group, alkylthio group, aryl group, aryloxy group, arylthio group, arylalkyl group, arylalkoxy group, arylalkylthio group, substituted amino group, substituted silyl group, monovalent heterocyclic group, or halogen atom represented by Re, Rf, Rg, Rh and Ri include the same groups as those shown as examples of the substituent which may be included in ring A, ring B and ring C. The arylalkenyl group means one in which the aryl described above is bonded to any one of carbon atoms of a lower alkenyl. The arylalkynyl group means one in which the aryl described above is bonded to any one of carbon atoms of a lower alkynyl. The substituted silyloxy group refers to a silyloxy group substituted with a substituent selected from an alkyl group and a phenyl group.

In the formula (6-4), it is preferred that Re and Rf are alkyl groups having 3 or less carbon atoms, alkoxy groups having 3 or less carbon atoms or alkylthio groups having 3 or less carbon atoms, and Rg is an alkyl group having 1 to 30 carbon atoms, an alkoxy group having 1 to 30 carbon atoms, or an alkylthio group having 1 to 30 carbon atoms.

In the group represented by the formula (5-1), a group in which Ar³ is represented by the formula (6-5) or a group represented by the formula (6-6) is preferred.

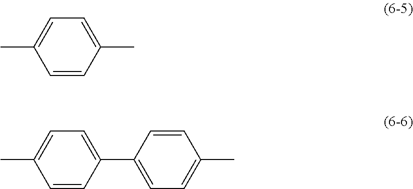

In the formula (6-5) and the formula (6-6), a benzene ring include in the group is preferably non-substituted, but may have 1 or more and 4 or less substituents. Those substituents may be the same or different from each other.

More preferred examples of the group represented by the formula (5-1) include groups represented by the formulas 141 to 143 shown below.

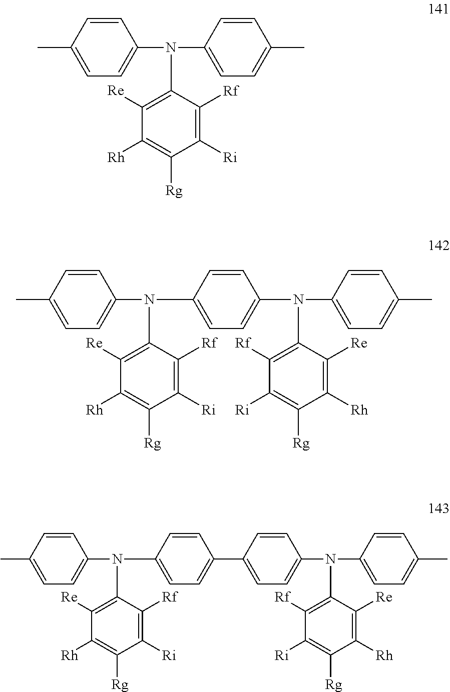

In the formulas 141 to 143, Re, Rf, Rg, Rh and Ri have the same meanings as defined above.

From the viewpoint of improvements in photoelectric conversion characteristics, still more preferred is the group represented by the formula (5-1), which is exemplified by the groups represented by the formulas (22) to (24) shown below.

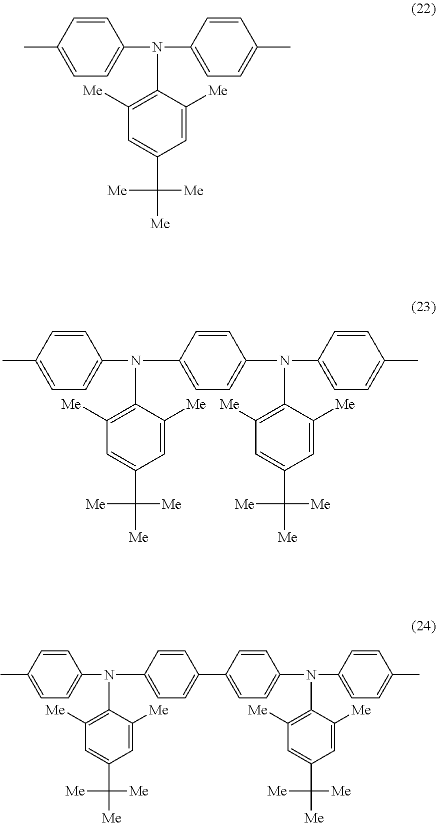

In the formula (5-2), ring D and ring E are the same or different and each represent an aromatic ring. A substituent may be included on ring D and ring E. From the viewpoint of stability of a polymer compound, an aromatic hydrocarbon ring is preferred, and a benzene ring is more preferred.

Preferred examples of the group represented by the formula (5-2) include groups represented by the formula (6-7).

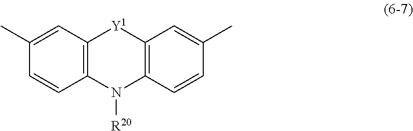

Examples of a monovalent group represented by R²⁰ include an alkyl group, an aryl group, an arylalkyl group, an alkenyl group, an alkynyl group, a substituted silyl group, an acyl group, a monovalent heterocyclic group and a substituted carboxyl group. Example of the alkyl group, aryl group, arylalkyl group, alkenyl group, alkynyl group, substituted silyl group, acyl group, monovalent heterocyclic group, and substituted carboxyl group include the same groups as those shown as examples of the substituent which may be included in ring A, ring B and ring C.

Among the polymer compound used in the present invention, a conjugated polymer is preferred from the viewpoint of improvements in electric charge transport properties in the case of being formed into a thin film, and photoelectric conversion characteristics. As used herein, the conjugated polymer means a polymer in which a non-localized π electron pair exists along the main chain backbone of a polymer. As non-localized electrons, an unpaired electron or a lone electron pair may sometimes take part in resonance in place of a double bond.

In the polymer compound used in the present invention, repeating units contained in the polymer compound may be connected to each other by non-conjugated units or the non-conjugated moieties thereof may be contained in the repeating units as long as electric charge transport characteristics are not impaired. Examples of the non-conjugated bond structure include structures represented by the general formulas shown below, and structures in which two or more structures among the structures represented by the general formulas shown below are used in combination.

In the formulas shown below, R(s) each have the same meaning as defined above. Ar(s) each represent an aromatic hydrocarbon ring or a heterocycle.

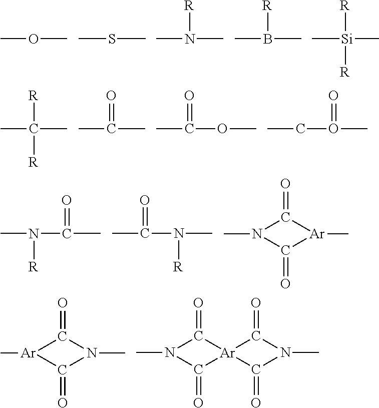

The polymer compound used in the present invention may be a random, alternating, block or graft copolymer, or a polymer having an intermediate structure thereof, for example, a random copolymer bearing a block property. From the viewpoint of obtaining high photoelectric conversion characteristics, a random copolymer bearing a higher block property, block or graft copolymer is more preferred than a completely random copolymer. Furthermore, in the polymer compound having an aromatic amine residue used in the present invention, one having the main chain being branched and having three or more terminal ends, and a dendrimer are also included.

From the viewpoint of an improvement in photoelectric conversion efficiency, it is preferred to contain a structural unit containing a structure represented by the formula (1) in the proportion of 0.1 mol % or more and 100 mol % or less, and more preferably 1 mol % or more and 70 mol % or less, based on all structural units in the polymer compound.

From the viewpoint of electric charge transport properties and injection properties, it is preferred to contain a structural unit containing a structure represented by the formula (1) in the proportion of 10 mol % or more and 100 mol % or less, and more preferably 30 mol % or more and 70 mol % or less, based on all structural units in the polymer compound.

It is more preferred to contain a repeating unit represented by the formula (4) in the proportion of 1 mol % or more and 99 mol % or less, and more preferably 50 mol % or more and 97 mol % or less, based on all repeating units in the polymer compound.

In a case where the polymer compound used in the present invention has at least one kind of a group represented by the formula (5-1) or a group represented by the formula (5-2), it is preferred to have a repeating unit composed of the group in the proportion of 0.01 mol % or more and 50 mol % or less, and more preferably 0.1 mol % or more and 30 mol % or less, based on all repeating units in the polymer compound from the viewpoint of electric charge transport properties and element characteristics.

The polymer compound used in the present invention preferably has a polystyrene-equivalent number average molecular weight of 2,000 or more, more preferably from 2×10³ to 1×10⁸, and still more preferably from 1×10⁴ to 1×10⁶, from the viewpoint of improvements in photoelectric conversion characteristics and film-forming properties. In the present description, a compound having a polystyrene-equivalent number average molecular weight of 2,000 or more is referred to as a polymer compound. A low-molecular compound is a compound having a single molecular weight, and the polystyrene-equivalent number average molecular weight is usually less than 2,000. The polymer compound used in the present invention may be a dendrimer, an oligomer or the like.

From the viewpoint of excellent photoelectric conversion characteristics, it is preferred that the polymer compound further contains a repeating unit represented by the formula (6):

—Ar¹—(CR⁸═CR⁹)—ₙ (6)

In the formula (6), Ar¹ represents an arylene group or a divalent heterocyclic group. R⁸ and R⁹ are the same or different and each represent a hydrogen atom, an alkyl group, an aryl group, a monovalent heterocyclic group, or a cyano group. n represents 0 or 1. The polymer compound used in the present invention may contain two or more kinds of repeating units represented by the formula (6).

Examples of the arylene group or divalent heterocyclic group represented by Ar¹ include the same groups as those of the arylene group or divalent heterocyclic group represented by Ar² to Ar⁵ described above. Examples of the alkyl group, aryl group or monovalent heterocyclic group represented by R⁸ and R⁹ include the same groups as those shown as examples of the substituent which may be included in ring A, ring B and ring C described above.

In the formula (6), n is preferably 0, and it is more preferred that n is 0 and Ar¹ is an arylene group.

The repeating unit represented by the formula (6) is desirably a repeating unit represented by the formula (6-1), or divalent aromatic amine residues such as carbazolediyl groups and triphenylaminediyl groups represented by the formulas 82 to 84, from the viewpoint of improving power generation characteristics.

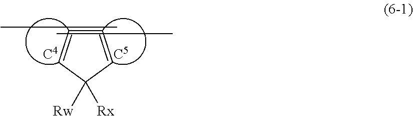

In the formula (6-1), ring C⁴ and ring C⁵ are the same or different and each represent an aromatic hydrocarbon ring optionally having a substituent, two bonds respectively exist on ring C⁴ and/or ring C⁵, and Rw and Rx are the same or different and each represent a hydrogen atom, an alkyl group, an alkoxy group, an alkylthio group, an aryl group, an aryloxy group, an arylthio group, an arylalkyl group, an arylalkoxy group, an arylalkylthio group, an alkenyl group, an alkynyl group, a substituted amino group, a substituted silyl group, an acyl group, an acyloxy group, an imine residue, an amide group, an acid imide group, a monovalent heterocyclic group, a substituted carboxyl group, a heteroaryloxy group, or a heteroarylthio group.

The aromatic hydrocarbon ring refers to a benzene ring or a fused aromatic hydrocarbon ring. The number of carbon atoms of such an aromatic hydrocarbon ring is from about 6 to 30, and preferably from about 6 to 15. The number of carbon atoms of the substituent is not included in the number of carbon atoms of the aromatic hydrocarbon group. Examples of such an aromatic hydrocarbon ring include a benzene ring, a naphthalene ring, an anthracene ring, a phenanthrene ring, a phenalene ring, a naphthacene ring, a triphenylene ring, a pyrene ring, a chrysene ring, a pentacene ring, a perylene ring, a pentalene ring, an indene ring, an azulene ring, a biphenylene ring, a fluorene ring and an acenaphthylene ring.

Examples of the alkyl group, alkoxy group, alkylthio group, aryl group, aryloxy group, arylthio group, arylalkyl group, arylalkoxy group, arylalkylthio group, alkenyl group, alkynyl group, substituted amino group, substituted silyl group, acyl group, acyloxy group, imine residue, amide group, acid imide group, monovalent heterocyclic group, substituted carboxyl group, heteroaryloxy group or heteroarylthio group in Rw and Rx include the same groups as those shown as the substituent which may be included in ring A, ring B and ring C.

Examples of the substituent, which may be included in ring C⁴ and ring C⁵, include a halogen atom, an alkyl group, an alkoxy group, an alkylthio group, an aryl group, an aryloxy group, an arylthio group, an arylalkyl group, an arylalkoxy group, an arylalkylthio group, an alkenyl group, an alkynyl group, a substituted amino group, a substituted silyl group, an acyl group, an acyloxy group, an imine residue, an amide group, an acid imide group, a monovalent heterocyclic group, a substituted carboxyl group, a heteroaryloxy group and a heteroarylthio group. Examples of the halogen atom, alkyl group; alkoxy group, alkylthio group, aryl group, aryloxy group, arylthio group, arylalkyl group, arylalkoxy group, arylalkylthio group, alkenyl group, alkynyl group, substituted amino group, substituted silyl group, acyl group, acyloxy group, imine residue, amide group, acid imide group, monovalent heterocyclic group, substituted carboxyl group, heteroaryloxy group, and heteroarylthio group include the same groups as those shown as the substituent which may be included in ring A, ring B and ring C.

Examples of the repeating unit represented by the formula (6-1) include repeating units represented by the general formulas shown below. Such repeating units may have at least one substituent selected from the group consisting of an alkyl group, an alkoxy group, an alkylthio group, an aryl group, an aryloxy group, an arylthio group, an arylalkyl group, an arylalkoxy group, an arylalkylthio group, an alkenyl group, an alkynyl group, a substituted amino group, a substituted silyl group, an acyl group, an acyloxy group, an imine residue, an amide group, an acid imide group, a monovalent heterocyclic group, a substituted carboxyl group, a heteroaryloxy group, a heteroarylthio group, and a halogen atom. Examples of the alkyl group, alkoxy group, alkylthio group, aryl group, aryloxy group, arylthio group, arylalkyl group, arylalkoxy group, arylalkylthio group, alkenyl group, alkynyl group, substituted amino group, substituted silyl group, acyl group, acyloxy group, imine residue, amide group, acid imide group, monovalent heterocyclic group, substituted carboxyl group, heteroaryloxy group, heteroarylthio group, and halogen atom include the same groups as those shown as the substituent which may be included in ring A, ring B and ring C. In the general formulas shown below, a bond in an aromatic hydrocarbon represents that it is possible to take any position.

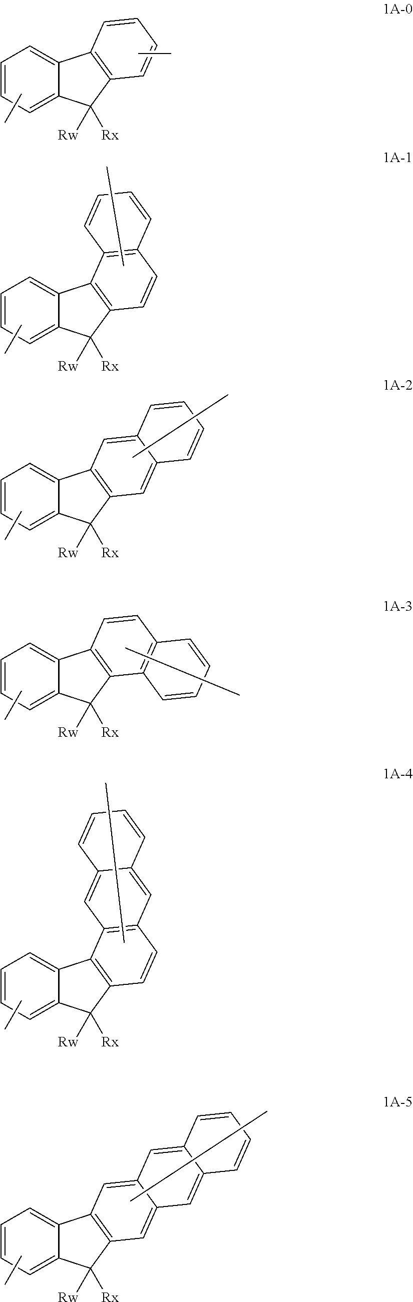

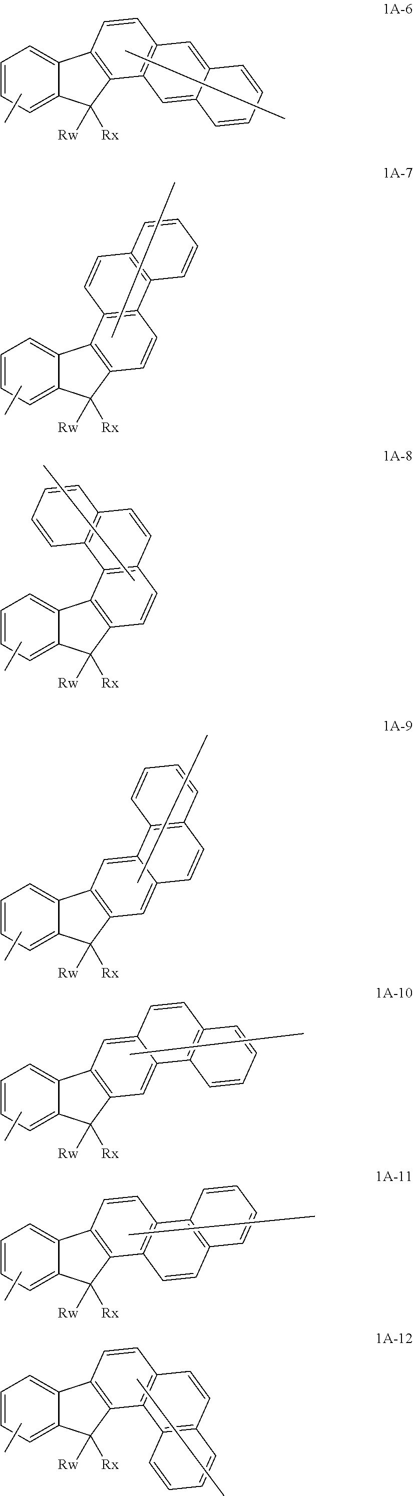

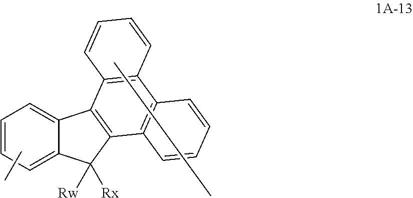

Among these repeating units, repeating units represented by 1A-0, 1A-1, 1A-2, and 1A-3 are preferred, and a repeating unit represented by 1A-0 is more preferred.

From the viewpoint of improvements in photoelectric conversion characteristics, it is preferred to further contain a repeating unit represented by the formula (6-3), in addition to a repeating unit represented by the formula (4), a group represented by the formula (5-1), and a group represented by the formula (5-2).

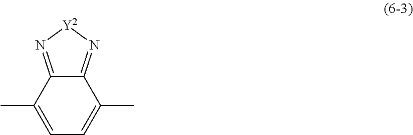

In the formula (6-3), Y² represents —O— or —S—. The repeating unit represented by the formula (6-3) contains two bonds existing on a 6-membered ring.

A method for producing the polymer compound used in the present invention will be described below.

One aspect of the method for producing the polymer compound used in the present invention is a method which comprises the step of polymerizing a compound represented by the formula (7) as a raw material. In other words, a polymer compound containing a repeating unit composed of the group represented by the formula (3) can be produced by polymerizing the compound represented by the formula (7) as a raw material.

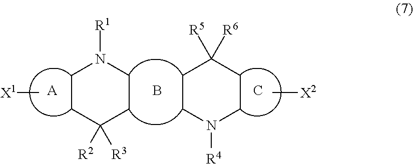

In the formula (7), ring A, ring B and ring C are the same or different and each represent an aromatic ring, R¹ and R⁴ are the same or different and each represent a monovalent group, R², R³, R⁵ and R⁶ are the same or different and each represent a hydrogen atom, an alkyl group, an aryl group, an arylalkyl group, an alkenyl group, or an alkynyl group, and X¹ and X² are the same or different and each represent a substituent which can be involved in polymerization. Examples of ring A, ring B, ring C, R¹, R², R³, R⁴, R⁵ and R⁶ include the same rings or groups as those described above.

In a case where the polymer compound used in the present invention contains a repeating unit other than a repeating unit composed of the group represented by the formula (3), the reaction may be carried out in the copresence of a monomer serving as a raw material of a repeating unit other than the repeating unit composed of the group represented by the formula (3).

The polymer compound used in the present invention can be produced by polymerizing a monomer having a substituent (polymerization active group) capable of being involved in the polymerization used as a raw material. The polymerization active group varies depending on a polymerization method and includes, for example, a formyl group, a phosphonium group, a halogen atom such as bromine, iodine or chlorine, a vinyl group, a halomethyl group, an acetonitrile group, an alkylsulfonyloxy group such as a trifluoromethanesulfonyloxy group, an arylsulfonyloxy group such as a toluenesulfonyloxy group, a boric acid residue (—B(OH)₂), a boric ester residue (for example, a group represented by —B(OQ)₂, wherein Q represents an alkyl group, an aryl group or the like, and two Q(s) may be bonded to form a ring) and the like. From the viewpoint of control of a molecular weight, control of a copolymerization ratio and the like, the number of the polymerization active groups is, preferably 2.

In a case where a vinylene group exists in the main chain, the polymer compound used in the present invention can be produced, for example, by the method described in JP-A-5-202355.

That is, examples of the method include [1] a polymerization method by the Wittig reaction of a compound having an aldehyde group with a compound having a phosphonium base, [2] a polymerization method by the Wittig reaction of compounds having an aldehyde group and a phosphonium base, [3] a polymerization method by the Heck reaction of a compound having a vinyl group with a compound having a halogen atom, [4] a polymerization method by the Heck reaction of compounds having a vinyl group and a halogen atom, [5] a polymerization method by the Horner-Wadsworth-Emmons reaction of a compound having an aldehyde group with a compound having an alkyl phosphonate group, [6] a polymerization method by the Horner-Wadsworth-Emmons reaction of compounds having an aldehyde group and an alkyl phosphonate group, [7] a polycondensation method by a dehydrohalogenation reaction of compounds having two or more halogenated methyl groups, [8] a polycondensation method by a sulfonium salt decomposition reaction of compounds having two or more sulfonium bases, [9] a polymerization method by the Knoevenagel reaction of a compound having an aldehyde group with a compound having an acetonitrile group, [10] a polymerization method by the Knoevenagel reaction of compounds having an aldehyde group and an acetonitrile group, and [11] a polymerization method by the McMurry reaction of compounds having two or more aldehyde groups and the like. The polymerization methods [1] to [11] described above are shown by the reaction schemes shown below.

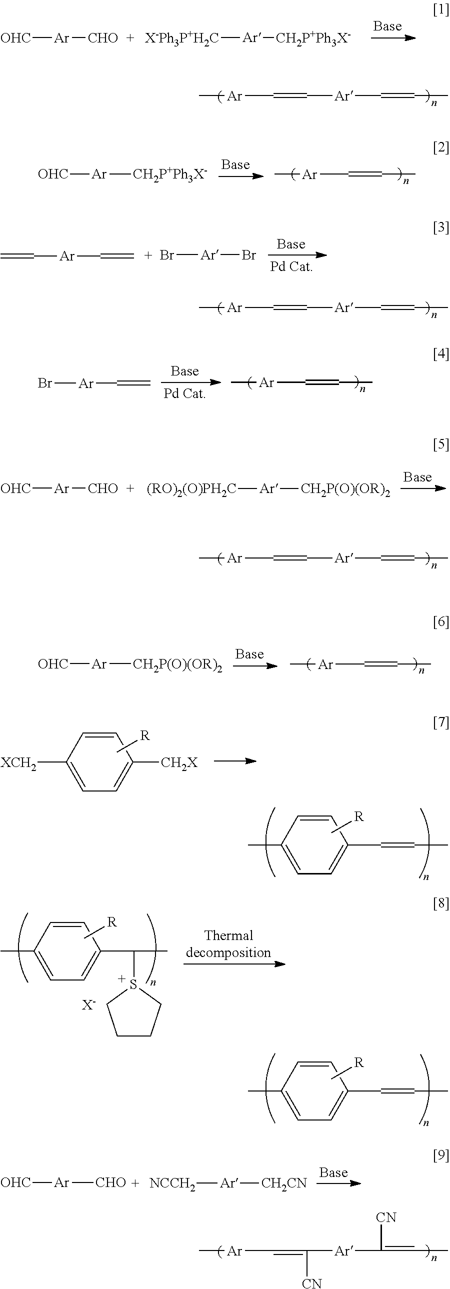

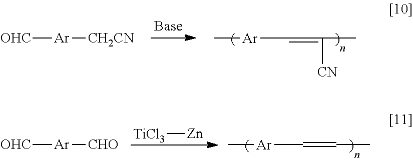

In a case where a vinylene group does not exist in the main chain, the polymer compound used in the present invention can be produced by polymerizing a monomer having a polymerization active group and, if necessary, other monomers. Examples of the method include [12] a polymerization method by the Suzuki coupling reaction, [13] a polymerization method by the Grignard reaction, [14] a polymerization method by the Stille coupling reaction, [15] a polymerization method by a Ni(0) catalyst, [16] a polymerization method by an oxidizing agent such as FeCl₃, [17] a method by a decomposition of intermediate polymer having a proper leaving group, an electrochemical oxidation polymerization method and the like. The polymerization methods [12] to [17] described above are shown by the reaction schemes shown below.

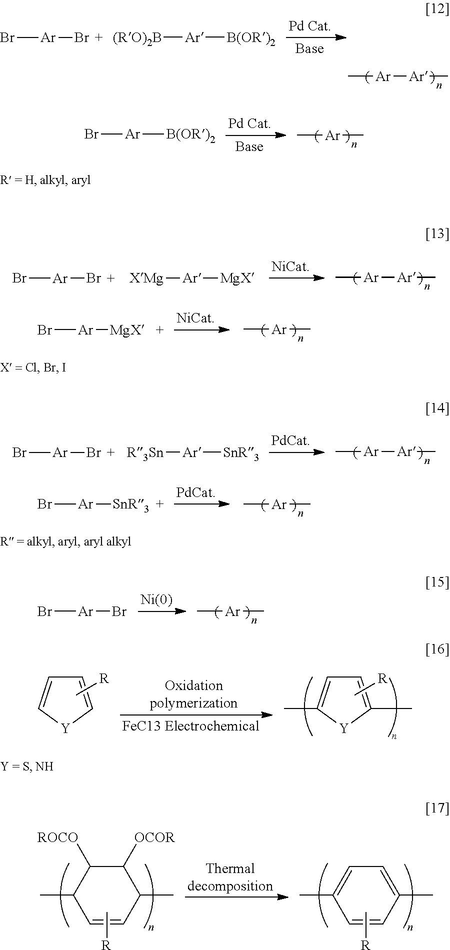

Among these polymerization methods, a polymerization method by the Wittig reaction, a polymerization method by the Heck reaction, a polymerization method by the Horner-Wadsworth-Emmons method, a polymerization method by the Knoevenagel reaction, a polymerization method by the Suzuki coupling reaction, a polymerization method by the Grignard reaction, a method using the Stille coupling reaction, and a polymerization method by a Ni(0) catalyst are preferred since it is easy to control a structure. Furthermore, a polymerization method by a Suzuki coupling reaction, a polymerization method by a Grignard reaction, and a polymerization method by a Ni(0) catalyst are preferred from the viewpoint of ease of availability of a raw material and convenience of polymerization reaction operation.

Among these methods, (i) a method using the Suzuki coupling reaction in which boric acid or a boric acid ester and a halogen atom, an alkyl sulfonate group, an aryl sulfonate group or an aryl alkyl sulfonate group are used in the presence of a palladium catalyst and a base; (ii) a method using the Yamamoto polymerization reaction in which a halogen atom, an alkyl sulfonate group, an arylsulfonate group or an aryl alkyl sulfonate group is coupled in the presence of a nickel(0); (iii) a method using the Stille coupling reaction in which a stannyl group and a halogen atom, an alkyl sulfonate group, an aryl sulfonate group or an aryl alkyl sulfonate group are used in the presence of a palladium catalyst; or (iv) the Grignard coupling method in which a halogenated magnesium is coupled with a halogen atom, an alkyl sulfonate group, an aryl sulfonate group or an aryl alkyl sulfonate group in the presence of a nickel catalyst is preferred since it is easy to obtain a polymer compound having a high molecular weight because of a high reaction yield and, when copolymerization is carried out, it is easy to control a reaction, for example, a copolymer having a monomer charge ratio which is the same as an original monomer charge ratio can be obtained. Among these methods, from the viewpoint of safety of a reagent, the Suzuki polymerization method and the Yamamoto polymerization method are more preferred. From the viewpoint of reactivity, among a halogen atom, an alkyl sulfonate group, an aryl sulfonate group or an aryl alkyl sulfonate group as a substituent (polymerization active group) which can be involved in polymerization, a chlorine atom, a bromine atom and an iodine atom are preferred, and a bromine atom is particularly preferred.

In the method for producing the polymer compound used in the present invention, a monomer is optionally dissolved in an organic solvent and then the obtained solution can be reacted at a melting point or higher and a boiling point or lower of the organic solvent using, for example, an alkali or a suitable catalyst. It is possible to use, as such a reaction method, known methods described, for example, in “Organic Reactions”, Vol. 14, pp. 270-490, John Wiley&Sons, Inc., 1965; “Organic Reactions”, Vol. 27, pp. 345-390, John Wiley&Sons, Inc., 1982; “Organic Syntheses”, Collective Volume VI, pp. 407-411, John Wiley&Sons, Inc., 1988; Chem. Rev., Vol. 95, p 2457 (1995); J. Organomet. Chem., Vol. 576, p 147 (1999); J. Prakt. Chem., Vol. 336, p 247 (1994); and Makromol. Chem., Macromol. Symp., Vol. 12, p 229 (1987).

The organic solvent varies depending on the compound and reaction used. However, it is generally preferred that the solvent used is sufficiently subjected to a deoxidation treatment and the reaction is allowed to proceed under an inactive atmosphere so as to suppress a side reaction. Similarly, it is preferred to carry out a dehydration treatment, except in the case of a reaction with water in a two-phase system like the Suzuki coupling reaction.

An alkali or a suitable catalyst is appropriately added so as to carry out the reaction. It may be selected according to the reaction used. Herein, the alkali or catalyst preferably sufficiently dissolves in the solvent used in the reaction. The method of mixing the alkali or catalyst includes a method in which a solution of an alkali or catalyst is slowly added while stirring a reaction solution under an inactive atmosphere such as argon or nitrogen, or to the contrary, a reaction solution is slowly added to a solution of an alkali or catalyst.

In a case where a photoelectric conversion element is prepared using the polymer compound described above, since purity thereof exerts an influence on characteristics, it is preferred to polymerize a monomer after purifying by a method such as a distillation, sublimation/purification or recrystallization method before polymerization. It is also preferred to subject to a purification treatment such as fractionation by reprecipitation/purification or chromatography after synthesis.

In the method for producing the polymer compound used in the present invention, each monomer may be reacted at a time, or separately mixed, if necessary.

If reaction conditions of the method for producing the polymer compound used in the present invention are described more specifically, the reaction is carried out using an alkali in an amount of 1 equivalent or more, preferably from 1 to 3 equivalents, based on a functional group of a monomer in the case of the Wittig reaction, Horner reaction, Knoevenagel reaction and the like. The alkali is not particularly limited and it is possible to use, for example, metal alcolates such as potassium-t-butoxide, sodium-t-butoxide, sodium ethylate, and lithium methylate; hydride reagents such as sodium hydride; and amides such as sodium amide. As the solvent, N,N-dimethylformamide, tetrahydrofuran, dioxane, toluene and the like are used. The temperature of the reaction is usually within a range from room temperature to about 150° C., which enables proceeding of the reaction. The reaction time is, for example, from 5 minutes to 40 hours, and it may be the time during which the polymerization sufficiently proceeds. It is not necessary that the reaction solution is not allowed to stand over along time after completion of the reaction, and thus the reaction time is preferably from 10 minutes to 24 hours. When the concentration in the case of the reaction is too low, efficiency of the reaction is low, whereas, when the concentration is too high, it becomes difficult to control the reaction. Therefore, the concentration may be appropriately selected within a range from about 0.01% by weight to a maximum dissolved concentration and is usually within a range from about 0.1% by weight to 20% by weight. In the case of the Heck reaction, a monomer is reacted in the presence of a base such as triethylamine using a palladium catalyst. Using a solvent having comparatively high boiling point such as N,N-dimethylformamide or N-methylpyrrolidone, the reaction temperature is from about 80 to 160° C. and the reaction time is from about 1 hour to 100 hours.

In the case of the Suzuki coupling reaction, using palladium[tetrakis(triphenylphosphine)] or palladium acetates or the like as a catalyst, inorganic bases such as potassium carbonate, sodium carbonate and barium hydroxide, organic bases such as triethylamine, and inorganic salts such as cesium fluoride are used in an amount of 1 equivalent or more, and preferably from 1 to 10 equivalents, based on the monomer and then the reaction is carried out. Using an inorganic salt in the form of an aqueous solution, the reaction may be carried out in a two-phase system. Examples of the solvent include N,N-dimethylformamide, toluene, dimethoxyethane and tetrahydrofuran. Although it varies depending on the solvent, the temperature used is suitably from about 50 to 160° C. Reflux may be carried out by raising the temperature to around boiling point of the solvent. The reaction time is from about 1 hour to 200 hours.

In the case of the Grignard reaction, there is shown a method in which a halide and metallic Mg are reacted in ether solvents such as tetrahydrofuran, diethylether and dimethoxyethane to give a Grignard reagent solution, and the Grignard reagent solution is mixed with a monomer solution prepared separately and, after adding a nickel or palladium catalyst while paying attention to excess reaction, the reaction is carried out while refluxing by raising the temperature. The Grignard reagent is used in an amount of 1 equivalent or more, preferably from 1 to 1.5 equivalents, and more preferably from 1 to 1.2 equivalents, based on the monomer. Also in the case of polymerization using a method other than these methods, the reaction can be carried out in accordance with a known method.

Examples of the method of reacting in the presence of a nickel catalyst include a method of polymerizing in the presence of the nickel(0) catalyst described above. Examples of the nickel catalyst include an ethylenebis(triphenylphosphine)nickel complex, a tetrakis(triphenylphosphine)nickel complex and a bis(cyclooctadienyl)nickel complex.

Examples of the reaction in the presence of a palladium catalyst include the Suzuki coupling reaction described above. Examples of the palladium catalyst include palladium acetate, a palladium[tetrakis(triphenylphosphine)]complex, a bis(tricyclohexylphosphine)palladium complex and a dichlorobis(triphenylphosphine)palladium complex.

The compound represented by the formula (7) is useful as a raw material for the production of the polymer compound used in the present invention.

Among the compounds represented by the formula (7), a compound represented by the formula (8) is preferred from the viewpoint of ease of synthesis.

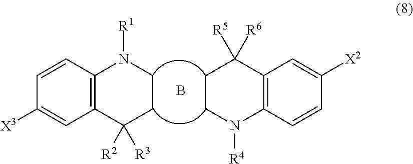

In the formula (8), ring B, R¹, R², R³, R⁴, R⁵, R⁶, X¹, X² have the same meanings as defined above.

The compound represented by the formula (8) can be synthesized from a compound represented by the formula (9).

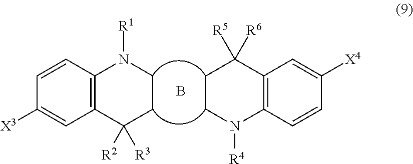

In the formula (9), X³ and X⁴ are the same or different and each represent a chlorine atom, a bromine atom, or an iodine atom. Further, ring B, R², R³, R⁴, R⁵, R⁶ have the same meanings as defined above.

The compound in which X¹ and/or X² in the formula (8) is a boric acid ester residue can be synthesized by replacing X³ and/or X⁴ of the compound represented by the formula (9) with a Grignard reagent or lithium, and then reacting it with a boric acid ester. Examples of such a boric acid ester include trimethylboric acid, triisopropylboric acid and 2-isopropoxy-4,4,5,5-tetramethyl-1,3,2-dioxaborolane. As described in J. Org. Chem., 60 (23), 7508 (1995), the compound represented by the formula (8) can be synthesized by reacting the compound represented by the formula (9) with diborate in the presence of a palladium catalyst and a base. Examples of this diborate include bis(pinacolate)diborane, bis(cathecolate)diborane, bis(neopentyl glycolate)diborane and bis(trimethylene glycolate)diborane.

The compound in which X¹ and/or X² in the formula (8) is —B(OH)₂ can be synthesized by a method of hydrolyzing the compound represented by the formula (8) in which X¹ and/or X² is a boric acid ester residue in the presence of an acid or a base.

The compound in which X¹ and/or X² in the formula (8) is a halogenated magnesium can be synthesized by reacting the compound represented by the formula (9) with magnesium.

For example, the compound in which X¹ and/or X² in the formula (8) is a trialkylstanyl group can be synthesized by replacing X³ and/or X⁴ of the compound represented by the formula (9) with a Grignard reagent or lithium, and then reacting it with a trialkyltin chloride. Examples of this trialkyltin chloride include trimethyltin chloride and tri-n-butyltin chloride.

The compound in which X¹ and/or X² in the formula (8) is an alkyl sulfonate group, an aryl sulfonate group or an aryl alkyl sulfonate group can be synthesized by replacing X³ and/or X⁴ of the compound represented by the formula (9) with a hydroxyl group, and then reacting with a corresponding sulfonic anhydride or a sulfonyl chloride in the presence of a base. Examples of this sulfonic anhydride include methanesulfonic anhydride, trifluoromethanesulfonic anhydride and benzenesulfonic anhydride. Examples of this sulfonyl chloride include methanesulfonyl chloride, trifluoromethanesulfonyl chloride and benzenesulfonyl chloride.

In the method in which X³ and/or X⁴ in the formula (9) is replaced with a hydroxyl group, it is possible to synthesize by oxidizing the compound obtained as described above and represented by the formula (8) in which X¹ and/or X² is —B(OH)₂, with a peroxide. Examples of this peroxide include hydrogen peroxide, m-chlorobenzeneperbenzoic acid and t-butyl hydroperoxide.

The compound represented by the general formula (9) described above can be synthesized by reacting a compound represented by the formula (10) with a halogenating agent. The compound represented by the formula (9) can be synthesized with satisfactory selectivity by selecting suitable reaction conditions.

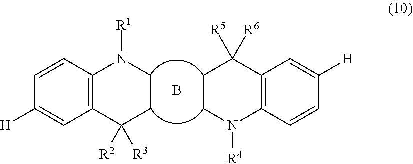

In the formula (10), ring B, R¹, R², R³, R⁴, R⁵, R⁶ have the same meanings as defined above.

Examples of the halogenating agent include N-halogeno compounds such as N-chlorosuccinimide, N-chlorophthalic acid imide, N-chlorodiethylamine, N-chlorodibutylamine, N-chlorocyclohexylamine, N-bromosuccinimide, N-bromophthalic acid imide, N-bromoditrifluoromethylamine, N-iodosuccinimide, and N-iodophthalic acid imide; halogen molecules such as fluorine, chlorine, and bromine molecules; and benzyltrimethylammonium tribromide. Among these groups, N-halogeno compounds are preferred.

Examples of the solvent used in the reaction include saturated hydrocarbons such as pentane, hexane, heptane, octane, and cyclohexane; unsaturated hydrocarbons such as benzene, toluene, ethylbenzene, and xylene; halogenated saturated hydrocarbons such as carbon tetrachloride, chloroform, dichloromethane, chlorobutane, bromobutane, chloropentane, bromopentane, chlorohexane, bromohexane, chlorocyclohexane, and bromocyclohexane; halogenated unsaturated hydrocarbons such as chlorobenzene, dichlorobenzene, and trichlorobenzene; alcohols such as methanol, ethanol, propanol, isopropanol, butanol, and t-butyl alcohol; carboxylic acids such as formic acid, acetic acid, and propionic acid; ethers such as dimethylether, diethylether, methyl-t-butyl ether, tetrahydrofuran, tetrahydropyran, and dioxane; amines such as trimethylamine, triethylamine, N,N,N′,N′-tetramethylethylenediamine, and pyridine; and amides such as N,N-dimethylformamide, N,N-dimethyl acetamide, N,N-diethylacetamide, N-methylmorpholine oxide, and N-methyl-2-pyrrolidone. These solvents can be used alone, or two or more kinds of them can be used in combination.

The temperature of the reaction is from −100° C. to the boiling point of the solvent, and preferably from about −20° C. to 50° C.

The compound represented by the formula (10) can be produced from a compound represented by the general formula (11) shown below in the presence of a base. For example, the compound represented by the formula (10) in which R¹ and/or R⁴ is an alkyl group can be produced by a nucleophilic substitution reaction to a halogenated alkyl in the presence of a base. Further, the compound represented by the formula (10) in which R¹ and/or R⁴ is an aromatic group (i.e., an aryl group, a monovalent aromatic heterocyclic group, the same shall apply hereinafter) can be produced by reacting with an aromatic iodide in the presence of copper and a base. As described in Angewandte Chemie, International Edition in English, (1995), 34 (12), 1348, the compound can also be produced by reacting a palladium catalyst, a base and a halogenated aromatic compound.

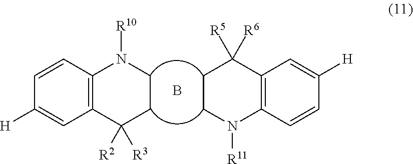

In the formula (11), R¹⁰ and R¹¹ are the same or different and each represent a hydrogen atom or a monovalent group, provided that at least one of R¹⁰ and R¹¹ is a hydrogen atom. Ring B, R², R³, R⁵, R⁶ have the same meanings as defined above. Examples of the monovalent group represented by R¹⁰ and R¹¹ include the same group as the monovalent group represented by R¹ described above.

The compounds represented by the formulas (10) and (11) can be produced by bringing a compound represented by the formula (12) into contact with an acid.

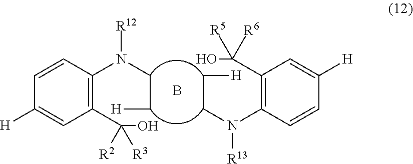

In the formula (12), R¹² and R¹³ are the same or different and each represent a hydrogen atom or a monovalent group. Ring B, R², R³, R⁵, R⁶ have the same meanings as defined above. Examples of the monovalent group represented by R¹² and R¹³ include the same group as the monovalent group represented by R¹ described above.

The acid may be a proton acid or a Lewis acid. Examples of the proton acid include sulfonic acids such as methanesulfonic acid, trifluoromethanesulfonic acid, and p-toluenesulfonic acid; carboxylic acids such as formic acid, acetic acid, trifluoroacetic acid, and propionic acid; and inorganic acids such as sulfuric acid, hydrochloric acid, nitric acid, and phosphoric acid. Among these proton acids, strong inorganic acids such as hydrochloric acid, sulfuric acid, and nitric acid are preferred. Examples of the Lewis acid include halogenated borides such as boron tribromide, boron trichloride, and a boron trifluoride ether complex; and halogenated metals such as aluminum chloride, titanium chloride, manganese chloride, iron chloride, cobalt chloride, copper chloride, zinc chloride, aluminum bromide, titanium bromide, manganese bromide, iron bromide, cobalt bromide, copper bromide, and zinc bromide. Among these Lewis acids, triphenylmethyl tetrafluoroborate is preferred. These Lewis acids can be used alone, or two or more kinds of them can be used in combination.

Although the acids described above may be used as a medium of the reaction, other solvents may also be used. Examples of the solvent used include saturated hydrocarbons such pentane, hexane, heptane, octane, and cyclohexane; halogenated saturated hydrocarbons such as carbon tetrachloride, chloroform, dichloromethane, chlorobutane, bromobutane, chloropentane, bromopentane, chlorohexane, bromohexane, chlorocyclohexane, and bromocyclohexane; halogenated unsaturated hydrocarbons such as chlorobenzene, dichlorobenzene, and trichlorobenzene; and nitrated compounds such as nitromethane and nitrobenzene. These solvents can be used alone, or two or more kinds of them can be used in combination.

The reaction temperature is from −50° C. to the boiling point of the solvent, and preferably from about 0 to 100° C.

In a case where the compound represented by the formula (10) in which at least one of R¹ and R⁴ is an aromatic group is synthesized, from the viewpoint of suppression of the side reaction, the method used is preferably a method in which the compound represented by the formula (11) in which R¹⁰ and R¹¹ are hydrogen atoms are produced using the compound represented by the formula (12) in which R¹² and R¹³ are hydrogen atoms, and then hydrogen atoms bonded with nitrogen atoms are substituted with aromatic groups.

The compound represented by the formula (12) can be produced by reacting the compound represented by the general formula: R¹⁴-M

wherein R¹⁴ represents an alkyl group, an aryl group, an arylalkyl group, an alkenyl group, or alkynyl group, and M represents lithium or halogenated magnesium, with a compound represented by the formula (13). Examples of the alkyl group, aryl group, arylalkyl group, alkenyl group, or alkynyl group represented by R¹⁴ include the same group as those shown as examples of the substituent which may be included in ring A, ring B and ring C.

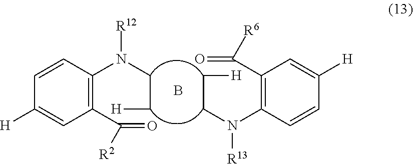

In the formula (13), ring B, R², R⁶, R¹², R¹³ have the same meanings as defined above.

The amount of the compound represented by the general formula: R¹⁴-M is preferably 4 equivalents or more based on the compound represented by the formula (13) when both R¹² and R¹³ in the formula (13) are hydrogen atoms. The amount of the compound represented by the general formula: R¹⁴-M is preferably 3 equivalents or more based on the compound represented by the formula (13) when either R¹² or R¹³ in the formula (13) is a hydrogen atom. The amount of the compound represented by the general formula: R¹⁴-M is preferably 2 equivalents or more based on the compound represented by the formula (13) when neither R¹² nor R¹³ in the formula (13) is a hydrogen atom.

The compound represented by the formula (12) can be produced by reacting a compound represented by the general formula: R¹⁵-M

wherein R¹⁵ represents an alkyl group, an aryl group, an arylalkyl group, an alkenyl group, or an alkynyl group, and M represents lithium or a halogenated magnesium, with a compound represented by the formula (14). Examples of the alkyl group, aryl group, arylalkyl group, alkenyl group or alkynyl group represented by R¹⁵ include the same groups as those shown as examples of the substituent which may be included in ring A, ring B and ring C.

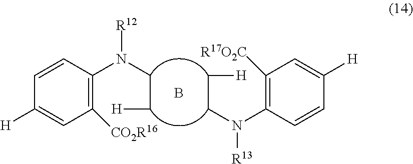

In the formula (14), R¹⁶ and R¹⁷ are the same or different and each represent an alkyl group, an aryl group, or an arylalkyl group. Ring B, R¹² and R¹³ have the same meanings as defined above. Examples of the alkyl group, aryl group or arylalkyl group represented by R¹⁶ and R¹⁷ include the same groups as those shown as the substituted which may be included in ring A, ring B and ring C.

The amount of the compound represented by the general formula: R¹⁵-M is preferably 6 equivalents or more based on the compound represented by the formula (14) when both R¹² and R¹³ in the formula (14) are hydrogen atoms. The amount of the compound represented by the general formula: R¹⁵-M is preferably 5 equivalents or more based on the compound represented by the formula (14) when either R¹² or R¹³ in the formula (14) is a hydrogen atom. The amount of the compound represented by the general formula: R¹⁵-M is preferably 4 equivalents or more based on the compound represented by the formula (14) when neither R¹² nor R¹³ in the formula (14) is a hydrogen atom.

Both the reaction in which the compound represented by the formula (12) is produced from the compound represented by the formula (13), and the reaction in which the compound represented by the formula (12) is produced from the compound represented by the formula (14) are preferably carried out under an atmosphere of an inert gas such as argon or nitrogen.

Examples of the solvent used in the reaction include saturated hydrocarbons such as pentane, hexane, heptane, octane, and cyclohexane; unsaturated hydrocarbons such as benzene, toluene, ethylbenzene, and xylene; and ethers such as dimethylether, diethylether, methyl-t-butylether, tetrahydrofuran, tetrahydropyran, and dioxane.

These solvents can be used alone, or two or more kinds of them can be used in combination.

The temperature of the reaction is from about −100° C. to the boiling point of the solvent, and preferably from −80° C. to 25° C.

A method for producing the compound represented by the formula (14) will be described below. The compound represented by the formula (14) can be produced from a compound represented by the formula (15), a compound represented by the formula (16) and a compound represented by the formula (17) in the presence of a catalyst containing at least one metal selected from the group consisting of palladium, nickel and copper.

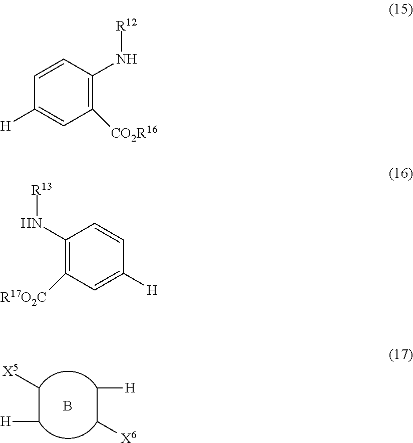

In the formulas (15) to (17), X⁵ and X⁶ are the same or different and each represent a chlorine atom, a bromine atom, an iodine atom, an alkyl sulfonate group, an arylsulfonate group, or an aryl alkyl sulfonate group. Ring B, R¹², R¹³, R¹⁶, R¹⁷ have the same meanings as defined above.

The compound can be produced by reacting an aromatic iodide with an aromatic amine in the presence of copper and a base as reaction conditions. As described in Angewandte Chemie, International Edition in English, (1995), 34(12), 1348, the compound can also be produced by reacting a palladium catalyst, a base and a halogenated aromatic compound.

From the viewpoint of ease of synthesis, the compound represented by the formula (15) and the compound represented by the formula (16) are preferably the same.

The compound represented by the formula (14) can also be produced from a compound represented by the formula (18), a compound represented by the formula (19) and a compound represented by the formula (20) in the presence of a catalyst containing at least one metal selected from the group consisting of palladium, nickel and copper.

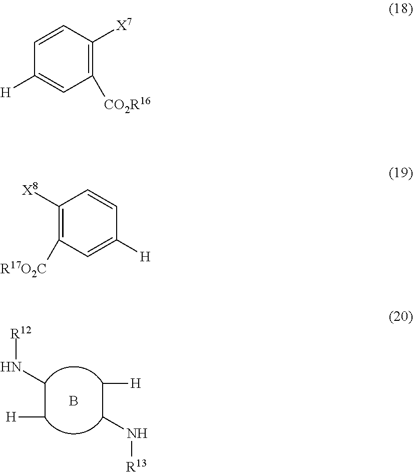

In the formulas (18) to (20), X⁷ and X⁸ are the same or different and each represent a chlorine atom, a bromine atom, an iodine atom, an alkyl sulfonate group, an aryl sulfonate group, or an aryl alkyl sulfonate group. Ring B, R¹², R¹³, R¹⁶, R¹⁷ have the same meanings as defined above.

The compound can be produced by reacting with an aromatic iodide in the presence of copper and a base as reaction conditions. As described in Angewandte Chemie, International Edition in English, (1995), 34(12), 1348, the compound can also be produced by reacting a palladium catalyst, a base and a halogenated aromatic compound.

From the viewpoint of ease of synthesis, the compound represented by the formula (18) and the compound represented by the formula (19) are preferably the same.

The photoelectric conversion element of the present invention includes a first electrode; a second electrode; a functional layer between the first electrode and the second electrode, the functional layer containing a polymer compound having an aromatic amine residue; and a porous semiconductor material containing a dye adsorbed thereon between the first electrode and the functional layer. The porous semiconductor material containing a dye absorbed thereon acts as a photoelectrode.

Among the dyes, a photosensitizing dye is preferred. The porous semiconductor material containing a dye absorbed thereon is preferably a porous semiconductor layer.

The photoelectric conversion element may include a dense layer between the first electrode layer and the functional layer, and the porous semiconductor material containing a dye adsorbed thereon may adhere onto a surface of the functional layer side of the dense layer. Further, the photoelectric conversion element may include an organic layer between the functional layer and the second electrode.

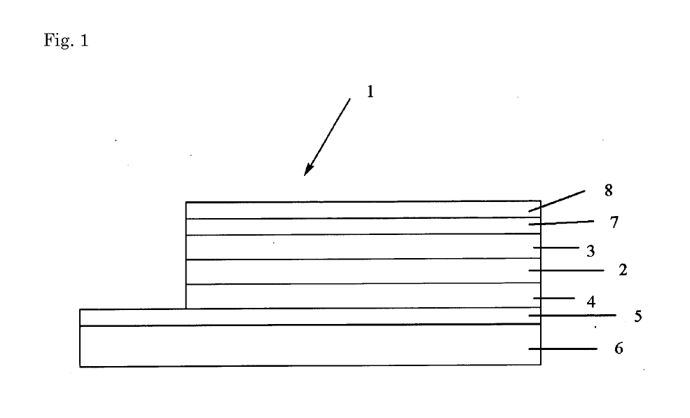

FIG. 1 is a diagram schematically showing a photoelectric conversion element 1 which is an embodiment of the photoelectric conversion element of the present invention. In the photoelectric conversion element 1, a first electrode 5, a dense layer 4, a functional layer 3, an organic layer 7, and a second electrode 8 are laminated on a substrate 6 in this order. The photoelectric conversion element includes a photoelectrode 2 containing a porous semiconductor material containing a dye absorbed thereon on a surface of the functional layer 3 side of the dense layer 4. A contact area between the photoelectrode 2 and the functional layer 3 is increased as much as possible by filling a portion of or all holes formed in the photoelectrode 2 with a polymer compound contained in the functional layer 3.

Each constituent element of the photoelectric conversion element 1 will be described together with each production method.

The substrate 6 may be a plate which exhibits flexibility or a plate which exhibits rigidity and a plate which does not undergo deformation in the step of producing a photoelectric conversion element is suitably used. The substrate 6 is preferably a substrate which exhibits high transmittance (transmittance is 80% or more at a wavelength of longer than 35 nm), and a glass and a plastic are usually used. Examples of the plastic include polyethylene terephthalate (PET), polyethylene naphthalate (PEN), polyphenylene sulfide (PPS), polycarbonate (PC), polypropylene (PP), polyimide (PI), triacetyl cellulose (TAC), syndiotactic polystyrene (SPS), polyallylate (PAR); cyclic polyolefin (COP) such as ARTON®, ZEONOR®, APEL®, or TOPAS®; and polyethersulfone (PES), polyetherimide (PEI), polysulfone (PSF), polyamide (PA). These plastics may be subjected to a treatment (treatment such as coating a surface with a gas-barrier substance) for improving gas barrier properties so as to prevent deterioration of a constituent material inside an element caused by moisture or oxygen. In a case where light is captured from those other than the substrate 6, an opaque plate may be used as the substrate 6. The substrate 6 has a role of protecting the functional layer 3, the dense layer 4 and the like.

The first electrode 5 functions as an electrode of taking out a photovoltaic force, together with the second electrode 8. The first electrode and/or the second electrode is made of at least one kind selected from the group consisting of an electrically conductive polymer, an oxide semiconductor and a metal. The first electrode 5 is formed, for example, by a thin film made of indium tin oxide (abbreviation: ITO), antimony tin oxide (abbreviation: ATO), tin oxide (SnO₂), zinc oxide, polyethylenedioxythiophene, polyaniline and the like. The first electrode 5 may be appropriately doped with additives and, for example, a thin film (FTO) made of SnO₂ doped with fluorine may be used as the first electrode 5. For example, the first electrode 5 is formed on the substrate 6 by a vacuum deposition method, a sputtering method, an ion plating method, a plating method, a spin coating method and the like.

The photoelectrode 2 is a layer in which a photosensitizing dye is adsorbed on a porous semiconductor. The porous semiconductor layer can be formed, for example, by firing semiconductor fine particles. The primary particle size of the semiconductor fine particles used in the photoelectrode 2 is from about 1 nm to 5,000 nm, and preferably from about 5 to 500 nm. The porous semiconductor layer may be formed using semiconductor fine particles entirely having nearly the same primary particle size, and the porous semiconductor layer may be formed using semiconductor fine particles having a different primary particle size. In the case of using the semiconductor fine particles having a different primary particle size, the wavelength of reflected light of fine particles varies with the primary particle size, and thus it is possible to reflect or scatter light in a wide wavelength range, and to improve photoelectric conversion efficiency. The shape of the semiconductor fine particles may be a generally spherical shape, a tubular shape or a hollow shape.

Examples of the material of the semiconductor fine particles include metal oxides such as titanium oxide, tin oxide, zinc oxide, iron oxide, tungsten oxide, zirconium oxide, hafnium oxide, strontium oxide, indium oxide, cerium oxide, yttrium oxide, lanthanum oxide, vanadium oxide, niobium oxide, tantalum oxide, gallium oxide, nickel oxide, strontium titanate, barium titanate, potassium niobate, and sodium tantalate; metal halides such as silver iodide, silver bromide, copper iodide, and copper bromide; metal sulfides such as zinc sulfide, titanium sulfide, indium sulfide, bismuth sulfide, cadmium sulfide, zirconium sulfide, tantalum sulfide, molybdenum sulfide, silver sulfide, copper sulfide, tin sulfide, tungsten sulfide, and antimony sulfide; metal selenides such as cadmium selenide, zirconium selenide, zinc selenide, titanium selenide, indium selenide, tungsten selenide, molybdenum selenide, bismuth selenide, and lead selenide; metal tellurides such as cadmium telluride, tungsten telluride, molybdenum telluride, zinc telluride, and bismuth telluride; metal phosphides such as zinc phosphide, gallium phosphide, indium phosphide, and cadmium phosphide; gallium arsenide, copper-indium-selenide, copper-indium-sulfide, silicon and germanium. Further, the material may be a mixture of two or more kinds of them. Examples of the mixture include a mixture of zinc oxide and tin oxide, a mixture of tin oxide and titanium oxide and the like.

Among them, metal oxides such as titanium oxide, tin oxide, zinc oxide, iron oxide, tungsten oxide, zirconium oxide, hafnium oxide, strontium oxide, indium, oxide, cerium oxide, yttrium oxide, lanthanum oxide, vanadium oxide, niobium oxide, tantalum oxide, gallium oxide, nickel oxide, strontium titanate, barium titanate, potassium niobate, sodium tantalate, a mixture of zinc oxide and tin oxide, and a mixture of tin oxide and titanium oxide are preferred since they are easily available at comparatively low price and a photosensitizing dye is easily adsorbed, and titanium oxide is more preferred.

The surface of semiconductor fine particles may be subjected to a chemical plating treatment using an aqueous titanium tetrachloride solution, or an electrochemical plating treatment using an aqueous titanium trichloride solution. This plating treatment enables an increase in the surface area of semiconductor fine particles, an increase in purity of crystallinity of the surface of semiconductor fine particles, covering impurities such as iron existing on the surface of semiconductor fine particles, and an enhancement in mutual connectivity and bonding property of semiconductor fine particles. Since a large amount of a photosensitizing dye can be adsorbed as the surface area of semiconductor fine particles increases, semiconductor fine particles having a large surface area are preferred. The surface area in a state where a porous semiconductor layer is formed on the substrate 6 is preferably 10 or more times, and more preferably 100 or more times larger than a project area where the porous semiconductor layer is projected on a place perpendicular to the thickness direction of the substrate 6. The upper limit thereof is usually about 1,000 times. The porous semiconductor layer may be a single layer or a multi-layer, and is usually a multi-layer. When the primary particle sizes of semiconductor fine particles used in each layer are different from each other, as described above, it is possible to reflect or scatter light in a wide wavelength range, and to improve photoelectric conversion efficiency.

The porous semiconductor layer can be formed, for example, by coating a slurry containing semiconductor fine particles using a coating method, followed by firing. Examples of the coating method include a method using a doctor blade, a squeegee, spin coating, dip coating, screen printing or the like. In the case of forming a film using the coating method, the average particle size in a dispersion state of semiconductor fine particles in the slurry is preferably from 0.01 nm to 100 μm. A dispersion medium into which the semiconductor fine particles are dispersed may be one capable of homogeneously dispersing the semiconductor fine particles therein, and water; and organic solvents, for example, alcohol solvents such as ethanol, isopropanol, t-butanol, and terpineol; ketone solvents such as acetone; and the like are used, and a mixed solvent of water and these organic solvents may also be used. The dispersion liquid may contain polymers such as polyethylene glycol; surfactants such as Triton-X; organic or inorganic acids such as acetic acid, formic acid, nitric acid, and hydrochloric acid; and chelating agents such as acetylacetone.

When the slurry is formed into a film by the coating method, the obtained film is then fired at a predetermined temperature. Firing is carried out at a temperature lower than the heatproof temperature of the substrate 6. For example, a glass substrate with a tin oxide film (first electrode 5) doped with fluorine is fired at about 450° C. to 550° C. Examples of the method of forming a porous semiconductor layer at comparatively low temperature include a hydrothermal method of subjecting to a hydrothermal treatment, a migration electrodeposition method in which a dispersion liquid of semiconductor particles dispersed therein is electrodeposited on a substrate, a pressing method in which a semiconductor paste is coated on a substrate, followed by drying and pressing and the like.

The dense layer 4 in contact with the first electrode 5 side of the porous semiconductor layer is preferably a dense semiconductor layer. The ratio of a primary particle size of semiconductor fine particles of the dense layer to a primary particle size of fine particles of a porous semiconductor material containing a dye absorbed thereon is preferably from 0.1 to 0.9, more preferably from 0.1 to 0.8, and still more preferably from 0.2 to 0.5.

For example, by coating a titania sol containing semiconductor fine particles having a particle size of about 10 nm dispersed therein on the first electrode 5 using a spin coating method to form the dense layer 4, a state where the functional layer 3 is contacted with the first electrode 5 is prevented, thus making it possible to suppress charge recombination which generates at an interface between the functional layer 3 and the first electrode 5, and to improve photoelectric conversion efficiency.

The film thickness of the functional layer 3 to be formed is from 15 μm to 10 nm, preferably from 10 μm to 20 nm, and still more preferably from 1 μm to 30 nm. From the viewpoint of an improvement in photoelectric conversion efficiency, the thickness is preferably from 90 nm to 40 nm.

As the photosensitizing dye to be adsorbed onto the porous semiconductor layer, materials exhibiting absorption in a visible light region and/or an infrared light region are suitably used, and one or two or more kinds of various metal complexes and organic dyes can be used. A photosensitizing dye having functional groups such as a carboxyl group, a hydroxyalkyl group, a hydroxyl group, a sulfone group, and a carboxyalkyl group in the molecule is preferred since it is quickly adsorbed onto the semiconductor layer described above. A metal complex is preferred as the photosensitizing dye since it is excellent in photoelectric conversion efficiency and durability. Examples of the metal complex used in the photosensitizing dye include metal phthalocyanines such as copper phthalocyanine and titanyl phthalocyanine, chlorophyll, hemin, and complexes of ruthenium, osmium, iron and zinc described in JP-A-1-220380 and JP-B-5-504023.

Specific examples of a ruthenium complex-based dye include cis-bis(isothiocyanate)bis(2,2′-bipyridyl-4,4′-dicarboxylate)-ruthenium (II)bis-tetrabutylammonium, cis-bis(isothiocyanate)bis(2,2′-bipyridyl-4,4′-dicarboxylate-ruthenium (II), tris(isothiocyanate)-ruthenium (II)-2,2′:6′,2″-terpyridine-4,4′,4″-tricarboxylic acid tris-tetrabutylammonium and cis-bis(isothiocyanate) (2,2′-bipyridyl-4,4′-dicarboxylate) (2,2′-bipyridyl-4,4′-dinonyl)ruthenium (II).

Examples of the organic dye used in the photosensitizing dye include a metal-free phthalocyanine dye, a cyanine dye, a merocyanine dye, a xanthene dye, a triphenylmethane dye, a squalirium dye and the like. Specific examples of the cyanine-based dye include NK1194 and NK3422 (both of which are manufactured by Nippon Kankoh Shikiso Kenkyusho Co., Ltd.). Specific examples of the merocyanine dye include NK2426 and NK2501 (both of which are manufactured by Nippon Kankoh Shikiso Kenkyusho Co., Ltd.). Examples of the xanthene dye include uranin, eosin, rose bengal, rhodamine B and dibromofluorescein. Examples of the triphenylmethane dye include Malachite Green and Crystal Violet. Examples of the cumarin dye include NKX-2677 (manufactured by Hayashibara Biochemical Laboratories, INC.), and specific examples thereof include a compound containing the structural unit shown below. Specific examples of an organic dye such as an indoline dye include a compound containing the structural unit shown below.

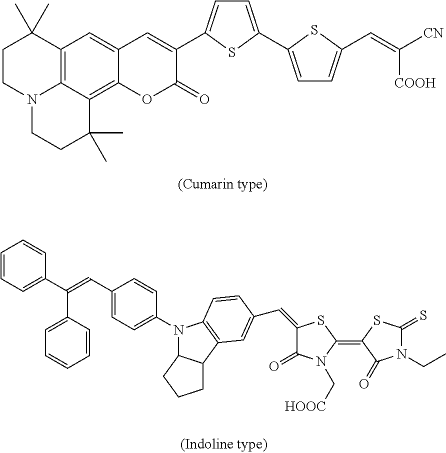

As a method of absorbing a photosensitizing dye onto a porous semiconductor layer, a method is used in which a substrate provided with a well dried porous semiconductor layer is immersed in a solution containing a photosensitizing dye for several hours. It is possible to use, as the solvent in which a photosensitizing dye is dissolved, acetonitrile, tertiary butyl alcohol, ethanol, methanol, dimethylformamide, dichloromethane, toluene, and a mixed solution thereof. Adsorption of the photosensitizing dye may be carried out at room temperature or under heating, or may be carried out while refluxing a solution containing the photosensitizing dye. Adsorption of the photosensitizing dye may be carried out before coating semiconductor fine particles on the substrate or after coating semiconductor fine particles on the substrate, and it is preferred to carry out after coating semiconductor fine particles on the substrate. In a case where a heat treatment is carried out after coating semiconductor fine particles on the substrate, adsorption of the photosensitizing dye is preferably carried out after the heat treatment. Since water is adsorbed on the surface of a semiconductor layer, a method of adsorbing a photosensitizing dye quickly as much as possible after the heat treatment so as to prevent adsorption of water is particularly preferred. When the unadsorbed photosensitizing dye existing without being adsorbed onto the semiconductor layer floats in the charge transport layer 3, the sensitizing effect may decrease. Therefore, it is more preferred to provide the step of removing the unadsorbed photosensitizing dye by washing, thereby making it possible to suppress the decrease of the sensitizing effect.

The photosensitizing dye to be adsorbed onto the semiconductor layer may be used alone, or a mixture prepared by mixing plural kinds of dyes. In a case where the photoelectric conversion element 1 is used as a photoelectrochemical cell, it is preferred to select the photosensitizing dye to be mixed so that it becomes possible to perform photoelectric conversion in a wide wavelength range as much as possible among the wavelength ranges of irradiating light such as sunlight. The amount of the photosensitizing dye to be adsorbed onto the semiconductor fine particles is preferably from 0.01 to 1 mmol based on 1 g of the semiconductor fine particles. When the amount of the dye is set within the above range, it is preferred that the sensitizing effect in the semiconductor layer is sufficiently obtained, thus suppressing the reduction of the sensitizing effect caused by floating of the dye which does not adhere onto the semiconductor layer.

The same photosensitizing dye may be adsorbed onto the entire semiconductor layer, or each different photosensitizing dye may be adsorbed onto each region after dividing the semiconductor layer into plural regions. For example, a photosensitizing dye having a different wavelength may be adsorbed onto each layer using a method in which the semiconductor layer is divided into three layers in the thickness direction, and then a photosensitizing dye capable of absorbing light having a wavelength of 300 nm to 500 nm is adsorbed onto a first layer, a photosensitizing dye capable of absorbing light having a wavelength of 500 nm to 700 nm is adsorbed onto a second layer, and a photosensitizing dye capable of absorbing light having a wavelength of 700 nm to 900 nm is adsorbed onto a third layer. Whereby, it is possible to realize the photoelectric conversion element 1 capable of performing photoelectric conversion of light having a wide wavelength range, and to improve photoelectric conversion efficiency.

It is preferred that a metal oxide film is formed on the photoelectrode 2 after adsorbing a photosensitizing dye onto the semiconductor layer. The film thickness of the metal oxide film is preferably a film thickness which enables exertion of a tunnel effect of electrons, and is for example, about several nm to several tens of nm. As the metal oxide film, for example, films made of one or two or more kinds selected from the group consisting of TiO₂, SnO₂, WO₃, ZnO, SiO₂, ITO, BaTiO₃, Nb₂O₅, In₂O₃, ZrO₂, Ta₂O₅, La₂O₃, SrTiO₃, Y₂O₃, Ho₂O₃, Bi₂O₃, CeO₂, and Al₂O₃ are used and, among these films, films made of Al₂O₃ and ZnO are suitably used. The metal oxide may be one doped with impurities, or a complex oxide or the like. By forming the metal oxide film, it is possible to prevent electrons in an oxide semiconductor injected from the photosensitizing dye from moving to the charge transport layer, thereby making it possible to improve photoelectric conversion efficiency.

The functional layer 3 can be formed by coating a liquid composition prepared by dissolving a polymer compound in an organic solvent on the dense layer 4 so as to cover the photoelectrode 2, followed by drying. In a case where the functional layer functions as a positive hole transport layer, the highest occupied molecular orbital (HOMO) level of a positive hole transport material used to form the positive hole transport layer is preferably the HOMO level or more of the photosensitizing dye. More preferably, the HOMO level of the positive hole transport material is 0.3 eV or more of the HOMO level of the photosensitizing dye. The lowest unoccupied molecular orbital (LUMO) level of the positive hole transport material is preferably the LUMO level or more of the photosensitizing dye. More preferably, the LUMO level of the positive hole transport material is 0.3 eV or more of the LUMO level of the photosensitizing dye. As the positive hole transport material (positive hole transporting material), a compound having a positive hole transport ability is used. Among these materials, a polymer compound having an aromatic amine residue is preferred. From the viewpoint of improvements in performances, the polymer compound used in the present invention is preferred. The positive hole transport ability is preferably 1×10⁻⁴ cm²/Vsec or more, and more preferably 1×10⁻³ cm²/Vsec or more in terms of positive hole mobility. From the viewpoint of improvements in performances of the photoelectric conversion element, 5×10⁻³ cm²/Vsec or more is preferred.

There is no limitation on a method of forming a functional layer. In a case where a polymer material is used as a material of the functional layer, a method of forming a film from a solution is given.

The solvent used to form a film from a solution is preferably a solvent which can uniformly dissolve or disperse a material of a functional layer, such as a positive hole transport material. Examples of such a solvent include chlorinated solvents such as chloroform, methylene chloride, 1,2-dichloroethane, 1,1,2-trichloroethane, chlorobenzene, and o-dichlorobenzene; ether solvents such as tetrahydrofuran and dioxane; aromatic hydrocarbon solvents such as toluene and xylene; aliphatic hydrocarbon solvents such as cyclohexane, methylcyclohexane, n-pentane, n-hexane, n-heptane, n-octane, n-nonane, and n-decane; ketone solvents such as acetone, methyl ethyl ketone, and cyclohexanone; ester solvents such as ethyl acetate, butyl acetate, and ethylcellosolve acetate; polyhydric alcohols and derivatives thereof, such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, and 1,2-hexanediol; alcohol solvents such as methanol, ethanol, propanol, isopropanol, and cyclohexanol; sulfoxide solvents such as dimethyl sulfoxide; and amide solvents such as N-methyl-2-pyrrolidone and N,N-dimethylformamide. These solvents can be used alone, or one or two or more kinds of them can be used in combination.

The optimum value of the film thickness of the functional layer varies depending on a material used, and may be selected so that the photoelectric conversion efficiency becomes a moderate value. It is necessary to have a thickness so as to prevent formation of pinholes. Too large thickness is not preferred since internal resistance of the element increases. It is necessary that the film thickness of such a functional layer is large enough to fill in the photoelectrode and, for example, the thickness is from 100 nm to 15 μm, more preferably from 500 nm to 13 μm, and still more preferably from 2 μm to 10 μm. To the coating solution described above, additives such as Li [CF₃SO₂]₂N, 4-t-butylpyridine and N(PhBr)₃SbCl₆ may be added. Examples of the coating method include a method using a doctor blade, a squeegee, spin coating, spray coating, dip coating, screen printing or the like. Drying may be carried out at normal temperature under normal pressure, or may be carried out using vacuum drying or the like. Penetration of the functional layer 3 into the photoelectrode 2 may be enhanced by decreasing a drying rate. In that case, a method of disposing the photoelectrode 2 after coating on a closed container or the like, or a method of disposing in a solvent atmosphere may be used.

The second electrode 8 may be provided so as to be in contact with the functional layer 3. For example, the second electrode 8 is made of at least one kind selected from the group consisting of an electrically conductive polymer, an oxide semiconductor and a metal. For example, the second electrode 8 is formed from a thin film made of Au, Ag, Al, Cu, Fe, Sb, Ti, TiO₂, Zn, Ni, ITO, ATO, FTO, polyethylenedioxythiophene, polyaniline and the like. For example, similar to the first electrode 5, it may be formed in advance in the form of a thin film on a substrate different from the substrate on which the first electrode 5 is formed. The substrate on which the thin film is formed is contact-bonded to the functional layer 3, thereby electrically connecting to the functional layer 3. The second electrode 8 is formed, for example, by a vacuum deposition method, a sputtering method, an ion plating method, a plating method and the like. In one present embodiment, although the second electrode 8 is laminated while being contacted with the functional layer 3, at least a portion of the second electrode may be disposed so as to intrude into the functional layer 3. Specifically, a bar-like second electrode may be inserted into the functional layer 3. It is preferred to insert the organic layer 7 into a space between the functional layer 3 and the second electrode 8.

Specifically, the organic layer 7 is preferably made of a polymer compound, and more preferably made of a polymer compound having high conductivity. By adjoining the organic layer made of a polymer compound having high conductivity to an anode and a functional layer, adhesion between the anode and the functional layer can be enhanced, and also efficiency of injection of holes (positive hole) from the functional layer into the electrode can be enhanced. Examples of the polymer compound include a polymer compound having a thiophenediyl group, a polymer compound having an anilinediyl group, a polymer compound having a pyrrolediyl group, a polymer compound having a fluorenediyl group and the like.

The organic layer used in the present invention can be formed by coating a solution containing a polymer compound and a solvent. As the coating method, the same method as that of forming a functional layer can be used. Examples of the function of the organic layer include a function to enhance efficiency of injection of positive holes into an anode, a function to prevent injection of electrons from a charge transport layer, a function to enhance transport ability of positive holes, a function to enhance smoothness in the case of deposition of an anode, a function to protect a functional layer from being eroded by a solvent in the case of forming an anode using a coating method, a function to reflect incident light from a cathode, a function to suppress deterioration of a charge transport layer and the like.

The film thickness of the organic layer used in the present invention is preferably from 1 nm to 100 μm, more preferably from 2 nm to 1,000 nm, still more preferably from 5 nm to 500 nm, and further preferably from 20 nm to 200 nm.

In order to ensure long-term stability of photoelectric conversion characteristics, the photoelectric conversion element 1 is preferably sealed with a sealing material. For example, the sides of the photoelectrode 2 and the functional layer 3 are coated with a sealing material. It is possible to use, as the sealing material, ionomer resins such as Himilan (manufactured by Mitsui DuPont Polychemical Co., Ltd.); glass frits; hot melt adhesives such as SX1170 (manufactured by Solaronix); adhesives such as Amosil 4 (manufactured by Solaronix); and BYNEL (manufactured by DuPont).

According to the photoelectric conversion element 1 of the present embodiment described above, since the functional layer 3 is solid, leakage of the functional layer can be prevented and a decrease in photoelectric conversion efficiency caused thereby can be prevented. Further, it is possible to suppress deterioration of photoelectric conversion characteristics caused by volatilization of the material contained in the functional layer. Furthermore, since a fully solid powder generation layer is used, sealing of a power generation element is not necessarily required and it is easy to realize flexibilization. In such a way, by constituting the functional layer using the polymer compound used in the present invention, a photoelectric conversion element having high reliability and high photoelectric conversion efficiency is realized.

### EXAMPLES

The present invention will be described in more detail by way of examples or the like, but the present invention is not limited to these examples. First, details of a method for synthesizing a polymer compound using the present invention will be described below.

### <Synthesis of Polymer Compound>

The compound A represented by the structural formula (A) shown below was synthesized in the following manner.

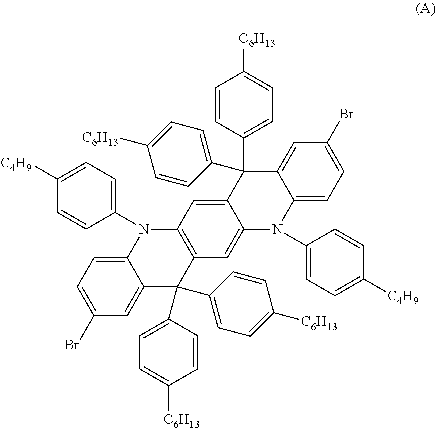

First, a compound represented by the structural formula (A-1) shown below was synthesized.

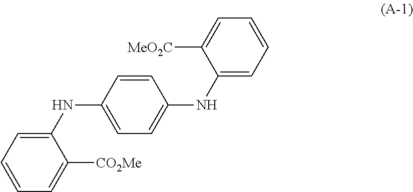

Into a 300 ml four-necked flask, 5.00 g of 1,4-dibromobenzene and 7.05 g of methyl anthranilate were charged and 100 ml of dehydrated toluene was added, followed by nitrogen bubbling for 1 hour. Then, 0.19 g of tris(dibenzylideneacetone)dipalladium, 0.24 g of tri(t-butyl)phosphine tetrafluoroborate and 10.36 g of cesium carbonate were added, followed by heating at 70° C. for 5 hours and further refluxing for 20 hours. The reaction solution was hot-filtered through a glass filter packed with 20 g of celite and washed with ethyl acetate. After the solvent was distilled off, the solution was washed in turn with an aqueous saturated sodium hydrogen carbonate solution, deionized water and saturated brine and then dried over sodium sulfate. The solvent was distilled off to obtain 5.49 g of a crude product. The aqueous phase was extracted three times with 100 ml of chloroform, washed with water and saturated brine, and then dried over sodium sulfate. The solvent was distilled off to further obtain 4.00 g of a crude product. The obtained crude products were combined and recrystallized from 30 ml of toluene to obtain 6.28 g of the compound (A-1).

### <Analytical Data>

*LC-MS

APPI-MS, positive 377 ([M+H]⁺, exact mass=376)

*¹H-NMR (300 MHz, CDCl₃)

δ 3.91 (6H, s), 6.72 (2H, t), 7.17-7.26 (6H, m), 7.31 (2H, t), 7.96 (2H, d), 9.42 (2H, s)

*¹³C-NMR (3000 MHz, CDCl₃)

δ 52.1, 111.8, 114.1, 117.1, 124.4, 131.9, 134.5, 136.9, 148.7, 169.3.

### <Synthesis of Compound A-2>

Next, a compound represented by the structural formula (A-2) was synthesized.

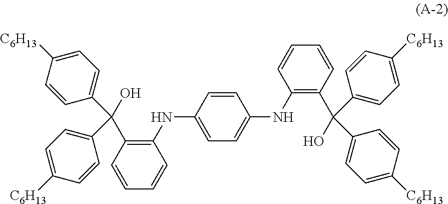

Into a 300 ml four-necked flask, 8.98 g of 1-bromo-4-n-hexylbenzene was charged, followed by nitrogen replacement. After dissolving in 90 ml of dehydrated THF and cooling to −78° C., n-butyllithium (1.6M hexane solution) was added dropwise over 10 minutes. After keeping the temperature for 2 hours, 2.00 g of the compound (A-1) was dissolved in 20 ml of dehydrated THF and added dropwise. After gradually warming to room temperature and stirring for 5 hours, 100 ml of water was added dropwise at 0° C. After liquid separation was carried out, the aqueous phase was extracted with 100 ml of ethyl acetate. The solution obtained by extraction was added to the oil phase, and the oil phase was washed in turn with water and saturated brine, and then the solvent was distilled off to obtain 8.86 g of a crude product (orange-red solid).

The obtained crude product was recrystallized from 50 ml of hexane to obtain 4.14 g of the compound (A-2).

### <Analytical Data>

*LC-MS

ESI, positive 999 ([M+K]⁺, exact mass=960)

*¹H-NMR (300 MHz, CDCl₃)

δ 0.88 (12H, t), 1.30 (24H, m), 1.60 (8H, m), 2.60 (8H, t), 4.74 (2H, brs), 5.68 (2H, brs), 6.55-6.63 (6H, m), 6.75 (2H, m), 7.03-7.26 (20H, m)

*¹³C-NMR (300 MHz, CDCl₃)

δ 14.4, 22.9, 29.3, 31.6, 32.0, 35.8, 82.4, 118.8, 120.2, 122.0, 127.9, 128.4, 130.3, 136.1, 137.7, 142.3, 143.3, 144.0.

### <Synthesis of Compound A-3>

Next, a compound represented by the structural formula (A-3) shown below was synthesized.

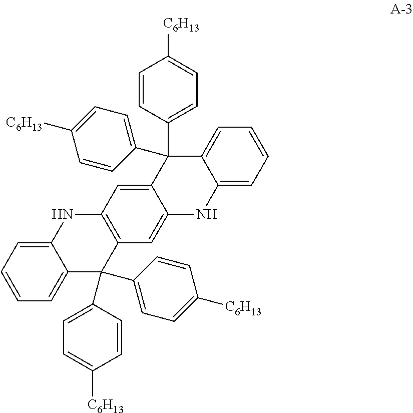

Into a 300 ml recovery flask, 8.00 g of the compound (A-2) was charged, followed by nitrogen replacement. After dissolving in 80 ml of acetic acid and cooling to 0° C., 2.8 ml of concentrated hydrochloric acid was added dropwise. After heating to room temperature, stirring for 5 hours and cooling again to 0° C., filtration and washing with water were carried out. The reaction solution was dissolved in 250 ml of toluene, made basic using an aqueous sodium hydroxide solution, washed in turn with an aqueous saturated sodium hydrogen carbonate solution, water and saturated brine, and then dried over sodium sulfate. Thereafter, the solvent was distilled off to obtain 13.75 g of a crude product. The obtained crude product was recrystallized from 50 ml of toluene to obtain 6.78 g of the compound (A-3).

### <Analytical Data>

*LC-MS

ESI, positive 963 ([M+K]⁺, exact mass=924)

*¹H-NMR (300 MHz, THF-d₈)

δ 0.90 (12H, t), 1.34 (24H, m), 1.55-1.62 (8H, m), 2.56 (8H, t), 6.37 (2H, s), 6.63-6.70 (6H, m), 6.87 (8H, d), 6.98-7.01 (10H, m), 7.87 (2H, s)

*¹³C-NMR (300 MHz, THF-d₈)

δ 13.7, 22.8, 29.5, 31.8, 32.0, 35.7, 56.2, 113.5, 115.2, 118.2, 126.8, 127.1, 127.3, 130.1, 130.4, 134.5, 140.4, 141.8, 144.6.

### <Synthesis of Compound A-4>

Next, a compound represented by the structural formula (A-4) shown below was synthesized.

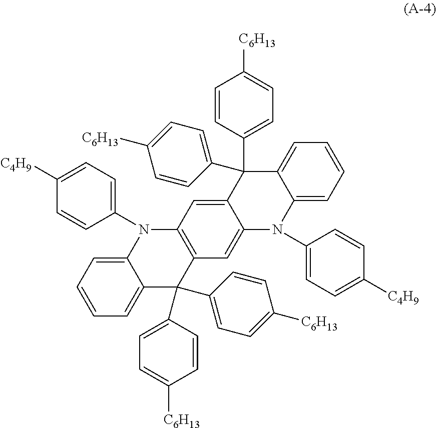

After nitrogen replacement, 9.90 g of the compound (A-3) and 4.94 g of 1-bromo-4-n-butylbenzene were charged into a 300 ml four-necked flask, and then dissolved in 150 ml of dehydrated toluene. After nitrogen bubbling for 20 minutes, 0.05 g of tris(dibenzylideneacetone)dipalladium, 0.03 g of tri(t-butyl)phosphine tetrafluoroborate and 0.30 g of sodium-t-butoxide were added, followed by refluxing for 10 hours. After cooling to 0° C., 100 ml of water was added, and liquid separation was carried out and the aqueous phase was extracted twice with 100 ml of toluene. The oil phases were combined, washed in turn with water and saturated brine, and then filtered through a glass filter packed with 60 g of silica gel. After washing with toluene, the solvent was distilled off to obtain 16.52 g of a crude product. The obtained crude product was recrystallized from 50 m of hexane added thereto and 50 ml of methanol was added, followed by filtration. The obtained solid was recrystallized from 50 ml of hexane to obtain 10.09 g of the compound (A-4).

### <Analytical Data>

*LC-MS

ESI, positive 1218 ([M+H]⁺, exact mass=1217)

*¹H-NMR (300 MHz, THF-d₈)

δ 0.62 (12H, t), 0.69 (6H, t), 1.00-1.34-17 (28H, 1.27-1.37 (12H, m), 2.26-2.35 (12H, m), 5.67 (2H, s), 5.93 (2H, d), 6.38-6.47 (8H, d), 6.56-6.61 (10H, m), 6.64 (8H, d), 6.79 (6H, d)

*¹³C-NMR (300 MHz, THF-d₈)

δ 15.5, 15.6, 24.6, 24.7, 31.3, 33.7, 33.9, 35.8, 37.6, 58.3, 116.0, 117.8, 121.2, 128.0, 129.1, 130.8, 138.5, 140.9, 142.2, 144.1, 145.0, 146.0.

### <Synthesis of Compound A>

Then, the compound A was synthesized. That is, into a 300 ml recovery flask, 10.09 g of the compound (A-4) was charged, followed by nitrogen replacement. After dissolving in 100 ml of chloroform and cooling to 0° C., a solution prepared by dissolving 2.87 g of NBS (N-bromosuccinimide) in 6 ml of dimethylformamide (DMF) was added dropwise over 20 minutes. An ice bath was removed and, after stirring for 7 hours and cooling to 0° C., 0.29 g of NBS was dissolved in 0.5 ml of DMF and the obtained solution was added dropwise. Furthermore, after stirring for 2.5 hours, 100 ml of water was added dropwise and liquid separation was carried out, and then the aqueous phase was extracted with chloroform. The oil phase was washed in turn with water and saturated brine, filtered through a glass filter packed with 150 g of silica gel and then washed with toluene. Then, the solvent was distilled off to obtain 12.48 g of a crude product. The crude products obtained from the aqueous phase and the oil phase were respectively recrystallized from 180 ml of hexane and 200 ml of hexane to obtain 9.55 g of the compound A.

### <Analytical Data>

*LC-MS

APCI, positive 1346 ([M+H]⁺, exact mass=1345)

*¹H-NMR (300 MHz, THF-d₈)

δ 0.90 (12H, t), 0.97 (6H, t), 1.30-1.45 (28H, m), 1.57-1.69 (12H, m), 2.56-2.61 (12H, m), 5.95 (2H, s), 6.16 (2H, brs), 6.70 (4H, d), 6.76 (8H, d), 7.01 (8H, d), 7.02 (2H, m), 7.05 (2H, m), 7.12 (4H, d)

*¹³C-NMR (300 MHz, THF-d₈)

δ 15.5, 15.6, 24.6, 24.7, 31.3, 33.6, 33.9, 35.7, 37.4, 37.6, 117.7, 129.4, 130.3, 132.0, 138.5, 142.7, 144.5.

### (Synthesis of Polymer Compound 1)

Under a nitrogen atmosphere, 1.01 g of the compound A, 0.40 g of 2,7-bis(1,3,2-dioxaboloran-2-yl)-9,9-di-n-octylfluorene, 0.5 mg of dichlorobis(triphenylphosphine)palladium, 0.10 g of trioctylmethylammonium chloride (manufactured by Aldrich under the trade name of “Aliquat336”) and 15 ml of toluene were mixed and then heated to 90° C. To this reaction solution, 4.1 ml of an aqueous 17.5% by weight sodium carbonate solution was added dropwise, followed by refluxing for 2 hours. After completion of the reaction, 10 mg of phenylboric acid was added, followed by refluxing for 4.5 hours. Then, an aqueous sodium diethyldithiacarbamate solution was added, followed by stirring at 80° C. for 3 hours. After cooling, the reaction solution was washed twice with 10 ml of water, washed twice with 10 ml of an aqueous 3% acetic acid solution, and then washed twice with 10 ml of water. The obtained solution was added dropwise in 120 mL of methanol and then a precipitate was obtained by filtration. This precipitate was dissolved in 25 mL of toluene and then purified by passing through a column in which active alumina was spread on silica gel. The obtained toluene solution was added dropwise to 120 ml of methanol, followed by stirring. The obtained precipitate was collected by filtration and then dried to obtain an amine type polymer compound. The yield of the obtained polymer compound 1 was 0.89 g. Further, the obtained polymer compound 1 showed a polystyrene-equivalent number average molecular weight of 1.0×10⁵ and a polystyrene-equivalent weight average molecular weight of 3.0×10⁵. The obtained polymer compound 1 showed a positive hole mobility of 6 to 8×10⁻² cm²/Vsec and a HOMO level of −4.7 eV.

### Example 1

On a transparent conductive glass substrate 6 on which a F (fluorine)-doped SnO₂ thin film (first electrode 5) is formed, a dense layer 4 was formed by coating titania sol PASOL HPW-10R manufactured by Catalysts&Chemicals Industries Co., Ltd. using a spin coating method and firing at 450° C. for 30 minutes. After firing, a titania paste (Solaronix Titananoxide D) manufactured by Solaronix was further coated by a spin coating method and then fired at 450° C. for 30 minutes to form a porous semiconductor material. Next, the porous semiconductor material described above was immersed in a solution prepared by mixing a mixed solvent of acetonitrile and tertiary butyl alcohol in a ratio of 1:1 (volume ratio) with a ruthenium dye (product name: Ruthenium 520-DN, HOMO level: −5.25 to −5.45 eV) manufactured by Solaronox. Whereby, a photoelectrode 2 made of a sensitizing dye-adsorbed mesoporous titanium dioxide having a film thickness of about 2 μm to 4 μm was formed. Then, 2% by weight of a polymer compound 1 was added to orthodichlorobenzene and dissolved by mixing with stirring at about 60° C. to 70° C. Then, 13 mM of (N-lithiotrifluoromethanesulfonimide) (Li[CF₃SO₂]₂N), 130 mM of 4-t-butylpyridine and 0.3 mM of tris(4-bromophenyl)aminium hexachloroantimonate) (N(PhBr)₃SbCl₆) were added, followed by mixing with stirring. The solution thus prepared was added dropwise on the photoelectrode 2 heated at about 60° C. and, after leveling for about 30 seconds, excess solution was removed by a spin coating method. Furthermore, drying was carried out in a container under an orthodichlorobenzene atmosphere to form a functional layer 3 which functions as a charge transport layer. Then, a HIL691 solution (manufactured by Plextronics under the trade name of Plexcore HIL691) was coated by a spin coating method to form an organic layer 7 having a film thickness of 70 nm. After drying, Au (second electrode 8) was deposited by a vacuum deposition method to produce a photoelectric conversion element of Example 1.

### Comparative Example 1

A photoelectric conversion element of Comparative Example 1, which is different from that of Example 1 only in a functional layer, was produced. Since the photoelectric conversion element was produced in the same manner as in the case of the photoelectric conversion element of Example 1, except for the functional layer, only the step of forming a charge transport layer is described. First, 2% by weight of poly(3-hexylthiophene) (HOMO level: −4.7 eV) was added to orthodichlorobenzene and dissolved by mixing with stirring at about 60° C. to 70° C. Then, 13 mM of Li[CF₃SO₂]₂N, 130 mM of 4-t-butylpyridine and 0.3 mM of N(PhBr)₃SbCl₆ were added, followed by mixing with stirring. The solution thus prepared was added dropwise on the photoelectrode 2 heated at about 60° C. and, after leveling for about 30 seconds, excess solution was removed by a spin coating method. Furthermore, drying was carried out in a container under an orthodichlorobenzene atmosphere to form a functional layer.

With respect to the photoelectric conversion elements of Example 1 and Comparative Example 1, photoelectric conversion characteristics were respectively measured. Short-circuit current density, open-circuit voltage, curve fill factor (fill factor), and photoelectric conversion efficiency measured with respect to each photoelectric conversion element are shown in Table 1.

TABLE 1
Photoelectric conversion characteristics

| | Short- | | | |
|---|---|---|---|---|
| | circuit | | | |
| | current | Open- | Curve fill | Photoelectric |
| | density | circuit | factor | conversion |
| | [mA/cm²] | voltage [V] | (fill factor) [—] | efficiency [%] |
| Example | 13.5 | 0.63 | 0.50 | 4.2 |
| Comparative | 9.9 | 0.57 | 0.38 | 2.2 |
| Example | | | | |

As is apparent from Table 1, in the photoelectric conversion element of Example 1, the short-circuit current density, open-circuit voltage and curve fill factor were improved when compared with the photoelectric conversion element of Comparative Example 1, because of using a solid type charge transport layer containing a polymer compound having a divalent aromatic amine residue, and thus the photoelectric conversion efficiency remarkably increased.

### INDUSTRIAL APPLICABILITY

The photoelectric conversion element of the present invention is free from leakage of a liquid material and has high photoelectric conversion efficiency, and is therefore industrially useful.

1. A photoelectric conversion element comprising a first electrode; a second electrode; a functional layer between the first electrode and the second electrode, the functional layer containing a polymer compound having an aromatic amine residue; and a porous semiconductor material containing a dye adsorbed thereon between the first electrode and the functional layer.

2. The photoelectric conversion element according to claim 1, comprising a dense layer between the first electrode layer and the functional layer, wherein the porous semiconductor material containing a dye adsorbed thereon adheres onto a surface of the functional layer side of the dense layer.

3. The photoelectric conversion element according to claim 1, comprising an organic layer between the functional layer and the second electrode.

4. The photoelectric conversion element according to claim 1, wherein the aromatic amine residue is one or more kinds of groups selected from the group consisting of a group in which at least one hydrogen atom has been removed from a structure represented by the formula (1), a group represented by the formula (5-1) and a group represented by the formula (5-2): 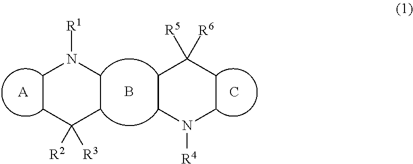
wherein ring A, ring B and ring C are the same or different and each represent an aromatic ring, R¹ and R⁴ are the same or different and each represent a monovalent group, and R², R³, R⁵ and R⁶ are the same or different and each represent a hydrogen atom or a monovalent group; 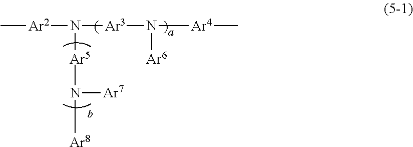
wherein Ar², Ar³, Ar⁴ and Ar⁵ are the same or different and each represent an arylene group or a divalent heterocyclic group, Ar⁶, Ar⁷ and Ar⁸ are the same or different and each represent an aryl group or a monovalent heterocyclic group, a and b are the same or different and each represent 0 or a positive integer, when plural Ar³(s) are present, they may be respectively the same or different, when plural Ar⁵(s) are present, they may be respectively the same or different, when plural Ar⁶(s) are present, they may be respectively the same or different, and when plural Ar⁷(s) are present, they may be respectively the same or different; and 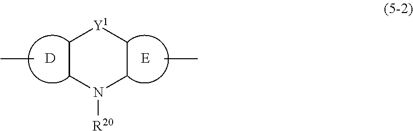
wherein ring D and ring E are the same or different and each represent an aromatic ring having a bond, Y¹ represents —O—, —S— or —C(═O)—, and R²⁰ represents a monovalent group.

5. The photoelectric conversion element according to claim 4, wherein R², R³, R⁵ and R⁶ are hydrogen atoms, alkyl groups, aryl groups, arylalkyl groups, alkenyl groups or alkynyl groups.

6. The photoelectric conversion element according to claim 5, wherein R², R³, R⁵ and R⁶ are groups represented by the formula (2): 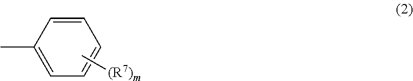
wherein R⁷ represents a halogen atom, an alkyl group, an alkoxy group, an alkylthio group, an aryl group, an aryloxy group, an arylthio group, an arylalkyl group, an arylalkyloxy group, an arylalkylthio group, an alkenyl group, an alkynyl group, a substituted amino group or a substituted silyl group, m represents an integer of from 0 to 5 and, when m is 2 or more, plural R⁷(s) may be the same or different from each other.

7. The photoelectric conversion element according to claim 4, wherein the group in which at least one hydrogen atom has been removed from a structure represented by the formula (1) is a group represented by the formula (3): 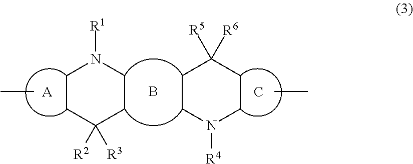
wherein ring A, ring B, ring C, R¹, R², R³, R⁴, R⁵ and R⁶ have the same meanings as defined above.

8. The photoelectric conversion element according to claim 7, wherein the polymer compound contains a repeating unit represented by the formula (4): 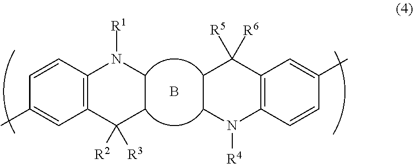
wherein ring B, R¹, R², R³, R⁴, R⁵ and R⁶ have the same meanings as defined above.

9. The photoelectric conversion element according to claim 8, wherein the polymer compound contains the repeating unit represented by the formula (4) and the group represented by the formula (5-1) or the group represented by the formula (5-2).

10. The photoelectric conversion element according to claim 1, wherein the polymer compound further contains a repeating unit represented by the formula (6):
—Ar¹—(CR⁸═CR⁹)—ₙ (6)
wherein Ar¹ represents an arylene group or a divalent heterocyclic group, R⁸ and R⁹ are the same or different and each represent a hydrogen atom, an alkyl group, an aryl group, a monovalent heterocyclic group or a cyano group, and n represents 0 or 1.

11. The photoelectric conversion element according to claim 1, wherein the polymer compound has a polystyrene-equivalent number average molecular weight of from 2×10³ to 1×10⁸.

12. The photoelectric conversion element according to claim 1, wherein the functional layer is in contact with the porous semiconductor material containing a dye adsorbed thereon.

13. The photoelectric conversion element according to claim 1, wherein the highest occupied molecular orbital level of the polymer compound is higher than the highest occupied molecular orbital level of the dye.

14. The photoelectric conversion element according to claim 1, wherein the lowest unoccupied molecular orbital level of the polymer compound is higher than the lowest unoccupied molecular orbital level of the dye.

15. The photoelectric conversion element according to claim 1, wherein the polymer compound has a positive hole mobility of 1×10⁻⁴ cm²/Vsec or more.

16. The photoelectric conversion element according to claim 1, wherein the first and/or second electrodes are made of at least one kind selected from the group consisting of an electrically conductive polymer, an oxide semiconductor material and a metal.

17. The photoelectric conversion element according to claim 1, wherein at least a portion of the second electrode is in contact with the functional layer.
